(19) United States
(12) Patent Application Publication
(10) Pub. No.: US 2025/0034520 A1
(43) Pub. Date: Jan. 30, 2025

(54) INCREASING DEVELOPMENTAL POTENTIAL OF HUMAN PREIMPLANTATION EMBRYOS BY REDUCING GENETIC INSTABILITY, ANEUPLOIDIES AND CHROMOSOMAL MOSAICISM

(71) Applicant: THE TRUSTEES OF COLUMBIA UNIVERSITY IN THE CITY OF NEW YORK, New York, NY (US)

(72) Inventor: Dietrich EGLI, New York, NY (US)

(21) Appl. No.: 18/909,710

(22) Filed: Oct. 8, 2024

Related U.S. Application Data
(63) Continuation of application No. PCT/US2023/018048, filed on Apr. 10, 2023.
(60) Provisional application No. 63/329,248, filed on Apr. 8, 2022.

(51) Int. Cl. C12N 5/073; C12N 9/22; C12N 15/11; C12N 15/90

(52) U.S. Cl. CPC C12N 5/0604; C12N 9/22; C12N 15/11; C12N 15/907; C12N 2310/20; C12N 2501/727

(57) ABSTRACT

Disclosed herein are agents, compositions and methods for increasing the developmental potential of human preimplantation embryos.

Disclosed herein are methods of reducing or decreasing replication abnormalities and/or aneuploidies in an embryo as well as increasing genome stability and/or developmental potential of an embryo by activating kinases and/or their signaling pathway in an oocyte including but not limited to ATR, WEE1, and CHK1.

Also disclosed herein are methods of reducing or decreasing replication abnormalities and/or aneuploidies in an embryo as well as increasing genome stability and/or developmental potential of an embryo using polynucleotides, polypeptides and/or agents which increase efficiency of the “fork reversion and repair” pathway and/or decrease detrimental outcomes such as fork collapse, translesion synthesis and/or gap formation.

Lastly disclosed herein are methods of reducing or decreasing replication abnormalities and/or aneuploidies in an embryo as well as increasing genome stability and/or developmental potential of an embryo using one or more polynucleotides or polypeptides including but not limited to CHEK1, WEE1, ETAA1, ATRX, BLM, BRCA2, CHD4, DNA2 (DNA2L), EXO1, FANCC, FANCG, FBH1 (FBX018), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCAI, ZRANB3, CDC6, CDT1, POLH, POLI, FANCD2, INO80, FANCB, ASH2L, FAM35A, XRCC3, BRIP1, and RNF168.

### CROSS REFERENCE TO RELATED APPLICATION

The present application is a continuation of International Application No. PCT/US2023/018048, filed Apr. 10, 2023, which claims priority to, and the benefit of, U.S. Provisional Application No. 63/329,248 filed on Apr. 8, 2022, all of which are incorporated by reference herein in their entirety.

### BACKGROUND

Human reproduction is remarkably inefficient, with abnormalities in the karyotype thought to be an important cause of developmental failure. Most early human embryos fail to progress through the earliest stages of development. It has been estimated that 30% of embryos fail to implant, 30% result in early pregnancy loss, and 15% lead to clinical miscarriages, leaving only about 25% of fertilized embryos contributing to a live birth (Fox et al. 2016). In subjects with subfertility, or in subjects with heritable mutations and undergoing preimplantation genetic diagnosis, the inefficiency of human development may be coupled with the presence of other infertility factors, presenting a nearly insurmountable obstacle.

Existing fertility treatments offer an imperfect solution to this problem by frequently directing treatment towards increasing the number of oocytes available for fertilization, often requiring several egg retrieval procedures. It has been estimated that on average, approximately 20 oocytes are required per one live birth, with variation occurring based on the age of the woman and other infertility factors (Lemmen et al. 2016). Therefore, in any given IVF cycle, only a fraction of embryos is eligible for implantation, the majority is excluded based on embryo grading/morphology. A poor morphology is associated with a reduced number of cells. Embryo loss increases the costs of IVF.

The number of IVF cycles failing to result in a pregnancy is staggering, or approximately 50%. IVF success rate is low, and the number of developmentally competent embryos is the key limiting factor. Aneuploidy is thought to be an important cause of developmental failure in humans, but its origins are not well known. In contrast to meiotic errors, which increase dramatically during maternal aging (Hassold and Hunt 2001), errors in mitosis occur independent of maternal age (Munne et al. 2002) and are thus relevant to all patients undergoing fertility treatments. The inefficiency of human development weighs most heavily at advanced maternal age, when fewer eggs are available, fewer are normal, and when less time is available to achieve a pregnancy. For younger patients, those who perform fertility preservation seek peace of mind, but no such security can currently be provided. Less than 40% of patients who attempt to achieve a pregnancy with cryopreserved oocytes do succeed (Cascante et al. 2022). The low efficiency of human development is a problem for the vast majority of patients undergoing fertility treatments.

There are no available treatments that increase the developmental potential of an oocyte or embryo, or avoid the emergence of new genetic abnormalities. The poor developmental potential of preimplantation embryos is specific to humans, and is not found in other mammals, such as mice. This suggests that it should be possible to increase the developmental potential of preimplantation embryos if the key differences between mice and man are understood. Due to a paucity of research on early human development, there is incomplete understanding of the mechanisms, and as a result, there is currently no treatment that can improve the efficiency of development potential in preimplantation embryos.

### SUMMARY

Disclosed herein are agents, compositions and methods for increasing the developmental potential of human preimplantation embryos.

Aneuploidy is a major cause of developmental failure in humans, but the origins of mitotic aneuploidies are not well understood. Shown herein is that DNA replication stress in the embryo caused genetic aneuploidies and developmental abnormalities. Human embryos acquire DNA damage during embryonic DNA replication, which is characterized by frequent replication fork stalling, replication fork slowing, and replication fork collapse, followed by incomplete replication at mitosis, chromosome breakage, micronucleation, and aneuploidy.

Also shown herein is that CHK1 inhibition specifically in the G2 phase resulted in a high degree of aneuploidy. These findings provide the basis for therapeutic intervention to prevent both genetic and developmental abnormalities. Specifically, strengthening the G2 checkpoint by activating one or more kinases and/or their signaling pathway including but not limited to ataxia telangiectasia and Rad3-related protein (ATR), WEE1, and checkpoint kinase 1 (CHK1) provides one basis of therapeutic intervention.

Thus, one embodiment of the current disclosure is a method of reducing or decreasing replication abnormalities and/or aneuploidies in an embryo comprising activating one or more kinases and/or their signaling pathway including but not limited to ATR, WEE1, and CHK1 in an oocyte.

A further embodiment of the current disclosure is a method of increasing genome stability and/or developmental potential of an embryo comprising activating one or more kinases and/or their signaling pathway including but not limited to ATR, WEE1, and CHK1 in an oocyte.

These methods can be accomplished by introducing the polynucleotide encoding the kinase or the kinase polypeptide or protein itself into an oocyte. The method can also be accomplished by introducing into an oocyte a polynucleotide encoding a polypeptide or polypeptide which activates the kinase and/or their signaling pathway, for example, ETAA1 and TOPBP1, both in their native and truncated forms, activate ATR.

These methods can also be accomplished by introducing an agent into an oocyte which activates the kinase and/or their signaling pathway, such as a small molecule or other pharmacological agents.

Additionally increasing efficiency of the “fork reversion and repair” pathway and decreasing or discouraging other detrimental outcomes such as fork collapse, translesion synthesis and gap formation provides a further basis of intervention. See FIG. 11J. Specifically, increasing or decreasing proteins and/or expression of genes which influence these pathways and outcomes provides a further basis for intervention.

Thus, one embodiment of the current disclosure is a method of reducing or decreasing replication abnormalities and/or aneuploidies in an embryo comprising introducing into an oocyte a polynucleotide which increases efficiency of the “fork reversion and repair” pathway and/or decreases fork collapse, translesion synthesis and/or gap formation.

A further embodiment of the current disclosure is a method of increasing genome stability and/or developmental potential of an embryo comprising introducing into an oocyte a polynucleotide which increases efficiency of the “fork reversion and repair” pathway and/or decreases fork collapse, translesion synthesis and/or gap formation.

A further embodiment of the current disclosure is a method of reducing or decreasing replication abnormalities and/or aneuploidies in an embryo comprising introducing into an oocyte a polypeptide which increases efficiency of the “fork reversion and repair” pathway and/or decreases fork collapse, translesion synthesis and/or gap formation.

A further embodiment of the current disclosure is a method of increasing genome stability and/or developmental potential of an embryo comprising introducing into an oocyte a polypeptide which increases efficiency of the “fork reversion and repair” pathway and/or decreases fork collapse, translesion synthesis and/or gap formation.

Yet a further embodiment of the current disclosure is a method of reducing or decreasing replication abnormalities and/or aneuploidies in an embryo comprising introducing into an oocyte an agent which increases efficiency of the “fork reversion and repair” pathway and/or decreases fork collapse, translesion synthesis and/or gap formation.

A further embodiment of the current disclosure is a method of increasing genome stability and/or developmental potential of an embryo comprising introducing into an oocyte an agent which increases efficiency of the “fork reversion and repair” pathway and/or decreases fork collapse, translesion synthesis and/or gap formation.

Also shown herein is that human embryos are more prone to acquire these abnormalities than mouse embryos. In contrast to humans, mice have higher replication fork restraint, replication fork reversion and processing ability, and mount stronger G2 checkpoint activation.

This largely protects the mouse embryo from aneuploidies. Thus, frequent chromosomal abnormalities are not an intrinsic feature to mammalian development, allowing comparative analyses. This finding provides a further basis for therapeutic intervention.

Analysis of differentially expressed genes between mice and humans involved in DNA repair revealed reduced expression of genes involved in fork reversion and processing as well as reduced expression of CHK1 and WEE1 checkpoint kinases, both of which inhibit entry into mitosis with unreplicated DNA. See FIGS. 11H and 11I. The differential expression of several genes points to a deficiency in replication fork recovery through accurate repair pathways and a deficiency in checkpoint control. These genes include but are not limited to CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, and XRCC2. Genes WRN, BRCA1, ZRANB3, CDC6, CDT, POLH, POLI, FANCD2, INO80, FANCB, ASH2L, FAM35A, XRCC3, BRIP1, and RNF168 are related to the listed genes and/or related to efficiency of the “fork reversion and repair” pathway and thus also can be used in the disclosed methods.

Thus one embodiment of the current disclosure is a method of reducing or decreasing replication abnormalities and/or aneuploidies in an embryo comprising introducing one or more polynucleotides selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof into an oocyte.

A further embodiment of the current disclosure is a method of increasing genome stability and/or developmental potential of an embryo comprising introducing one or more polynucleotides selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN. BRCA1. ZRANB3. CDC6, CDT1, POLH, POLI, FANCD2, INO80, FANCB, ASH2L, FAM35A, XRCC3, BRIP1, RNF168, and combinations thereof into an oocyte.

Yet a further embodiment of the current disclosure is a method of reducing or decreasing replication abnormalities and/or aneuploidies in an embryo comprising introducing one or more polypeptides selected from the group consisting of CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN. BRCA1, ZRANB3, CDC6, CDT1, POLH, POLI, FANCD2, INO80, FANCB, ASH2L, FAM35A, XRCC3, BRIP1, RNF168, and combinations thereof into an oocyte.

A further embodiment of the current disclosure is a method of increasing genome stability and/or developmental potential of an embryo comprising introducing one or more polypeptides selected from the group consisting of CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof into an oocyte.

These methods can also be accomplished by introducing an agent into an oocyte which activates one or more the genes, such as a small molecule or other pharmacological agents.

Thus a further embodiment of the current disclosure is a method of reducing or decreasing replication abnormalities and/or aneuploidies in an embryo comprising introducing one or more agents which activate and/or increase the expression of one or more genes selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6. CDT1, POLH, POLI, FANCD2, INO80, FANCB, ASH2L, FAM35A, XRCC3, BRIP1, RNF168, and combinations thereof into an oocyte.

A further embodiment of the current disclosure is a method of increasing genome stability and/or developmental potential of an embryo comprising introducing one or more agents which activate and/or increase the expression of one or more genes selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof into an oocyte.

In some embodiments, the embryo and the oocyte are human.

In some embodiments, a combination of therapeutic interventions/methods can be utilized. For example, a polynucleotide, polypeptide and/or which activates a kinase can be used in conjunction with a polynucleotide, polypeptide and/or agent which increases the expression of one of the differentially expressed genes involved in DNA repair.

In some embodiments, the polynucleotide is mRNA. In some embodiments, the polynucleotide is DNA.

In some embodiments, one polynucleotide or polypeptide is introduced. In some embodiments, more than one polynucleotide or polypeptide is introduced. In some embodiments, two or more polynucleotides or polypeptides are introduced. In some embodiments, five or more polynucleotides or polypeptides are introduced.

In some embodiments, a biological equivalent of the one or more polynucleotides or polypeptides is introduced.

In some embodiments, a variant, mutant, fragment, homologue, or paralogue of the one or more polynucleotides or polypeptides is introduced.

In some embodiments, the polynucleotide or polypeptide is introduced into the oocyte at the time of fertilization by the sperm.

In some embodiments, the method is carried out during an in vitro fertilization procedure.

The disclosure also provides for kits.

### BRIEF DESCRIPTION OF THE DRAWINGS

For the purpose of illustrating the invention, certain embodiments of the invention are depicted in drawings. However, the invention is not limited to the precise arrangements and instrumentalities of the embodiments depicted in the drawings.

FIG. 1 shows a schematic of the therapeutic concept and workflow. The treatment involves the application of one factor or a combination of factors at fertilization or during the first cell cycle, to stabilize the genome. The following steps are consistent with the state of the art: monitoring of development and a decision to implant based on morphology and/or molecular characterization after a trophectoderm biopsy.

The following Figures (FIGS. 2-10) establish the concept of DNA replication stress in the embryo as a cause of genetic aneuploidies and developmental abnormalities. This provides the basis for therapeutic intervention to prevent both genetic and developmental abnormalities.

FIG. 2. Markers of DSBs in human embryos arise in the first G2 phase in an ATR dependent manner

FIG. 2A is a schematic of developmental stages of a human embryo. FIG. 2B is a graph of the quantification of nuclear foci at indicated stages for γH2AX. Horizontal bar: mean. The number of cells and embryos (in parenthesis) is indicated. FIG. 2C is a graph of the quantification of nuclear foci at indicated stages for RAD51. Horizontal bar: mean. The number of cells and embryos (in parenthesis) is indicated. FIG. 2D is a graph of the quantification of γH2AX foci at the 1-cell stage 18 h into the first cell cycle with the indicated inhibitor. Horizontal bar: median. FIG. 2E is a graph of the quantification of RPA32I nuclear foci at indicated stages. Horizontal bar: mean. The number of cells and embryos (in parenthesis) is indicated. FIG. 2F is a graph of RPA32 p-533 foci at of blastomere at indicated stages. FIG. 2G is a graph of the quantification of foci for RPA32 p-S4S8 in individual blastocyst stage embryos. FIG. 2H is a graph of the quantification of 53BPI nuclear foci at indicated stages. Horizontal bar: mean. The number of cells and embryos (in parenthesis) is indicated. FIG. 2I is a graph of the frequency of 53BP1 bodies depending on developmental stage. Statistical analysis using student's t-test. 53BP1 bodies follow the formation of foci with a delay of one or more cell cycles. FIG. 2J is a graph of the percentage of cells in day5 blastocysts with 53BP1 bodies (>1 mm diameter) or 53BP1 foci (<1 mm diameter). Horizontal bar indicates average. FIG. 2K is graphs of the analysis of RNAseq data from GSE44183 (Xue et al., 2013) for 53BP1, RNF8 and RNF168 from the one-cell stage to embryonic genome activation in human embryos. Individual datapoints are individual single cells. **p<0.001 using one-way Anova. FIG. 2L is a graph of the quantification of abnormal nucleation in zygotes, cleavage and blastocyst embryos. Horizontal bar indicates mean. Scale bars=5 m. ****p<0.0001, ***p<0.001, **p<0.001, *p<0.05. Statistical analysis using one-way ANOVA.

FIG. 3. Human and mouse zygotes complete DNA replication in late G2 phase

FIG. 3A is a schematic of the first cell cycle in human zygotes. Dark shading indicates canonical S-phase with EdU positive signal. The lighter shading indicates DNA synthesis in G2 phase. FIG. 3B is a graph of the timing of mitotic entry (hours post ICSI or activation) in the presence of aphidicolin (Aph) or aphidicolin plus CHK1 inhibitor (CHKi) or aphidicolin plus ATR inhibitor (ATRi) or controls. Number of oocytes is indicated. Statistical analysis using one-way ANOVA. FIG. 3C is a graph of the timing of mitotic entry in human zygotes and parthenotes after CHK1 inhibition in G2 phase using either LY2603618 or AZD7762. Statistical analysis using one-way ANOVA. FIG. 3D shows the number of aneuploid chromosomes per cell in control 2-cell embryos, or after treatment in late G2 phase with either CHK1 inhibitor alone or in combination with Aph until entry into mitosis.

FIG. 4. Asymmetric sister fork progression, low replication fork speed, and G2 replication in the first cell cycle

FIG. 4A is a schematic of the timing of the first cell cycle in mouse. The lines outside the circle indicate the timing of application of the indicated compound. FIG. 4B is a graph of the quantification of DNA replication fork track speed in mouse. FIG. 4C is a graph of quantification of DNA replication fork speed in humans. FIG. 4D is quantification of fork speed in humans after PARP inhibition using Olaparib. FIG. 4E is a table of the quantification of sister fork asymmetry. ****P=0.00001, ***P=0.0001 using Fisher's exact test. FIG. 4F is a graph of the replication track ratio first/second label through preimplantation development. FIG. 4G shows the replication track ratio first/second label through preimplantation development with PARP inhibition using Olaparib in mice. FIG. 4H shows the replication track ratio first/second label through preimplantation development with PARP inhibition using Olaparib in humans. FIG. 4I is a graph of the percent of mouse embryos progressing from G2 to the first mitosis depending on timing of aphidicolin application. The total numbers of eggs are indicated for each condition. Each dot represents an independent experiment consisting of at least ten embryos. Wee1i=Wee1 inhibitor. Statistical analysis using student's t-test. FIG. 4J is a graph of the timing of mitotic entry in mouse controls and after exposure to indicated replication inhibitors in late G2 phase. FIG. 4K is a graph of the timing of mitotic entry in mouse controls and after Atr or Chk1 inhibition. Horizontal bars: median. n=number of embryos.

FIG. 5. Spontaneous chromosome breaks in day3 embryos map to gene poor areas and result in chromosome loss

FIG. 5A is a graph of the frequency of euploid and aneuploid cells in day3 cleavage stage embryos. FIG. 5B is a chart of the quantification of error types. FIG. 5C shows the quantification of gains and losses, displayed as a ratio in spontaneous and Cas9-induced aneuploidies. ‡‡ data from meiotic segregation errors from (Franasiak et al., 2014), ‡ data from (Zuccaro et al., 2020). ***p<0.001, **p<0.01 using Fisher's exact test.

FIG. 6. Spontaneous chromosome breaks in day3 embryos map to gene poor areas and result in chromosome loss

FIG. 6A is a chart of the localization of chromosomal break sites. FIG. 6B is the quantification of gene density at chromosomal break sites in comparison to 50 randomly selected sites. Mean and standard deviation is indicated. Dotted line indicates genome average. Mann-Whitney test ****p<0.0001. FIG. 6C shows the break sites on specific chromosomes with reciprocal or mirroring representation in the same embryo.

FIG. 7. Micronuclei are spontaneous mitotic aneuploidies involving chromosome breakage

FIG. 7A shows the ploidy of micronuclei isolated from day3 or arrested multicell day5 embryos. FIG. 7B is a chart of the quantification of the type of abnormalities seen in isolated micronuclei. FIG. 7C shows the analysis of gene density at sites of chromosome breakage. Mean and standard deviation is indicated. Dotted line is the genome average. Mann-Whitney test ****p<0.0001.**p<0.01.

FIG. 8. Genome instability is independent of embryonic genome activation

FIG. 8A shows the size of long transcripts and of intergenic areas overlapping with 22 fragile regions. FIG. 8B shows the expression of long transcripts (RPKM) at fragile sites in human preimplantation embryos. No bar is shown when expression is undetectable. * indicates genes with mitotic DNA synthesis in somatic cells (Ji et al., 2020, Macheret et al., 2020). Data from GSE36552 (Yan et al., 2013). FIG. 8C is a quantification of micronuclei in control IVF embryos and embryos treated with RNA polymerase inhibitor from day1-day3. Statistical analysis using one-way ANOVA. ***p<0.001, ****p<0.0001. FIG. 8D shows the gene density at break sites before and at EGA compared to simulated random breakage. Statistical analysis using Mann-Whitney test. *p<0.05, ****p<0.0001. FIG. 8E shows the frequency of aneuploid cells through the first three cell divisions.

FIG. 9. Gene poor regions require G2 DNA synthesis

FIG. 9A is a chart of the quantification of types of errors. FIG. 9B is a chart of the genomic characteristics of chromosomal break sites. FIG. 9C shows the density of protein-coding genes and size of affected transcription units at break sites. Intergenic sites are at 0 transcript length on the Y-axis. Break sites at long transcripts or gene rich-regions are indicated. Sites labeled in green are concordant with spontaneous breakage in IVF embryos. FIG. 9D is a comparison of gene density at spontaneous, aphidicolin-induced and random sites ****p<0.0001 using a Mann-Whitney test. FIG. 9E shows vertical bars indicating break point position at the RALYL locus on chromosome 8, in parthenogenetically activated embryos (independent events red and orange with aphidicolin added at 15.5 h post activation), fertilized zygotes (purple with aphidicolin added at 18 h post ICSI), and spontaneous breakage in day3 blastomeres (blue).

FIG. 10. Induced chromosome fragility at late replicating regions compromises developmental potential in mouse embryos

FIG. 10A is a graph of the quantification of abnormal anaphase figures for indicated conditions. Statistical analysis using one way ANOVA. FIG. 10B is the quantification of aneuploidies per cell for indicated conditions. G2 checkpoint inhibitor includes ATR inhibition (10 samples) and CHKi (17 samples). Statistical analysis using one way ANOVA. FIG. 10C is a chart of the quantification of the types of chromosomal abnormalities at the 2-cell stage. Fragmentation is defined as 3 or more copy number transitions on the same chromosome. FIG. 10D shows the percentage of gains and losses. FIG. 10E shows the percent blastocyst development at indicated conditions. The 119 ATR and CHK1 inhibition samples consist of 57 ATR and 62 CHK1 inhibition. Statistical analysis using one way ANOVA. FIG. 10F shows the analysis of embryo quality through quantification of cell number. FIG. 10G shows the analysis of embryo quality through quantification ICM cell number (Pou5f1 positive cells). Statistical analysis using student's t-test, bars indicate mean and standard error. FIG. 10H shows the analysis after APH treatment in G2 phase of the first cell cycle and the quantification of DNA damage markers at the 4-8 cell stage, 48 h post activation. Statistical analysis using one way ANOVA. FIG. 10I shows the analysis after APH treatment in G2 phase of the first cell cycle and is the quantification of micronuclei per embryos at 48 h post activation. No micronucleation was observed in controls. Statistical analysis using student's t-test. ****p<0.0001, ***p<0.001, **p<0.01, *p<0.05.

The following Figure establishes differences in replication fork stability and checkpoint response between mouse and human embryos. Differentially expressed factors between mouse and human embryos identify therapeutically relevant factors.

FIG. 11. Response to unreplicated DNA differs between mouse and human embryos

FIG. 11A is a schematic of timing of application of aphidicolin and/or MRE11 inhibitor in the first G2 phase in mice. FIG. 11B is the corresponding quantification of γH2AX and RPA32 foci after 6 h of aphidicolin (Aph) incubation per haploid nucleus in mice. FIG. 11C is a schematic of timing of application of aphidicolin and/or MRE11 inhibitor in the first G2 phase in humans. FIG. 11D is the corresponding quantification of γH2AX and RPA32 foci after 6 h of aphidicolin (Aph) incubation per haploid nucleus in human. The species is indicated below each panel. Horizontal bars indicate averages. Statistical analysis using student's t test.*p<0.05,**p<0.01, ***p<0.001, ****p<0.0001. FIG. 11E shows MRE11 endonuclease or exonuclease dependence of γH2AX and Rad51 foci formation at forks stalled by aphidicolin. Exonuclease inhibition dramatically increases the number of foci while endonuclease inhibition completely abrogates it. Statistical analysis using one-way ANOVA. FIG. 11F is the quantification of RAD51 foci in the MRE11 inhibitor study in human parthenotes FIG. 11G is the quantification of γH2AX foci in the MRE11 inhibitor study in human parthenotes. FIG. 11H shows the transcript abundance in human and mouse 1-cell stage embryos. 69 transcripts involved in DSB repair and replication fork stability represented in two datasets containing both mouse and human data GSE44183 (Xue et al., 2013) and GSE18290 (Xie et al., 2010) were analyzed, and the 25 genes shown were differentially expressed in both datasets. Shown are data from Xue et al. Statistical analysis using Welch's t-test, *p<0.05, **p<0.01, ***p<0.001 ****p<0.0001. FIG. 11I shows additional data on the transcript abundance in human and mouse 1-cell stage embryos in transcripts involved in DSB repair and replication fork stability represented in both mouse and human data GSE44183 (Xue et al., 2013). Shown are data from Xue et al. Statistical analysis using Welch's t-test, *p<0.05, **p<0.01, ***p<0.001 ****p<0.0001. FIG. 11J is a model for different outcomes of stalled replication forks and postreplicative ssDNA gaps in human and mouse embryos. Genes labeled in green are showing higher transcript levels in 1-cell human embryos than in mouse, while genes labeled in red show lower transcript levels. PARP inhibition by Olaparib is thought to result in increased replication fork progression but also more gap formation. For more details on factors involved in the repair of stalled forks, see review by (Joseph et al., 2020). Red arrows indicate paths to aneuploidy. Whether and how postreplicative ssDNA gaps contribute to aneuploidy is not well understood and is thus labeled with a question mark. The molecular nature of the replication-stalling lesion is not known. FIG. 11K shows expression of checkpoint kinase transcripts in human and mouse 1-cell stage embryos. Data from (Xie et al., 2010). Statistical analysis using Welch's t-test, ***p<0.001. FIGS. 11L-11O show the requirement for kinase in the first G2 phase at the mouse 1-cell stage. FIG. 11L shows the experimental schematic. FIG. 11M is the quantification of aneuploidies depending on indicated condition. FIG. 11N is a schematic of treatment. FIG. 11O shows the timing of mitotic entry after indicated condition. FIG. 11P is a graph of the developmental competence after treatment in first G2 phase with indicated inhibitors. FIG. 11Q shows a comparison of the frequency of markers of DSBs and repair in human and mouse embryos at the blastocyst stage. Note the significantly lower level of all makers in mouse embryos. Scale bars=5 mm. Statistical analysis using one way ANOVA. ***p<0.0001, **p<0.001, *p<0.01.

### DETAILED DESCRIPTION

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

The term “agent” as used herein means a substance that produces or is capable of producing an effect and would include, but is not limited to, chemicals, pharmaceuticals, drugs, biologics, small molecules, antibodies, nucleic acids, peptides, and proteins.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the terms “nucleic acid sequence” and “polynucleotide” and “nucleic acid” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto to polypeptide sequences. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also includes a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” or homology (equivalence or equivalents) to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

The term “variant” of a protein, peptide or polypeptide means a protein, peptide or polypeptide that has an amino acid sequence which differs by one or more amino acids from the polypeptide sequence.

A “variant” of a polynucleotide sequence is defined as a polynucleotide sequence which differs from the reference polynucleotide sequence by having deletions, insertions and/or substitutions.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “vector” includes any genetic element, such as a plasmid, phage, transposon, cosmid, chromosome, artificial chromosome, virus, or virion, which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene.

The term “expression vector” or “expression construct” or “construct” means any type of genetic construct containing a nucleic acid in which part or all of the nucleic acid encoding sequence is capable of being transcribed. In some embodiments, expression includes transcription of the nucleic acid, for example, to generate a biologically active polypeptide product or inhibitory RNA from a transcribed gene.

With respect to transfected host cells, the term “transfection” is used to refer to the uptake of foreign DNA by a cell, and a cell has been “transfected” when exogenous DNA has been introduced inside the cell membrane. A number of transfection techniques are generally known in the art. See, e.g., Graham et al., Virology 52:456 (1973), Sambrook et al., Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically active polypeptide or production of a biologically active nucleic acid such as an RNA, has been introduced.

The term “embryo” refers to the early stage of development of a multicellular organism. In general, in organisms that reproduce sexually, embryonic development refers to the portion of the life cycle that begins just after fertilization and continues through the formation of body structures, such as tissues and organs. Each embryo starts development as a zygote, a single cell resulting from the fusion of gametes (i.e., fertilization of a female egg cell by a male sperm cell). In the first stages of embryonic development, a single-celled zygote undergoes many rapid cell divisions, called cleavage, to form a blastula. In some embodiments, the embryo is human. In some embodiments, the embryo is a non-human animal.

Aneuploidy is thought to be an important cause of developmental failure in humans. In contrast to meiotic errors, which increase dramatically during maternal aging, errors in mitosis occur independent of maternal age, and are thus relevant to all patients undergoing fertility treatments. Due to a paucity of research on early human development, there is incomplete understanding and no available treatment that can improve the efficiency of fertility treatments.

Shown herein is that chromosomal aneuploidies in the early embryo are due to abnormalities in DNA replication. DNA replication is the first act of growth in the embryo, even before any gene expression occurs. DNA replication during the first cell divisions is characterized by frequent DNA replication fork stalling, replication fork slowing, replication fork collapse, and the formation of DNA breaks. Unreplicated DNA and DNA breaks result in both whole-chromosome as well as segmental aneuploidies and in embryo attrition.

Interestingly, human embryos are much more prone to acquire these abnormalities than mouse embryos. Thus, frequent chromosomal abnormalities are not an intrinsic feature to mammalian development. Shown herein is the examination of genes in replication fork recovery, double strand break repair and checkpoint control. Differentially expressed genes were identified that may be limiting to genome stability in humans. These genes include but are not limited to CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, and XRCC2.

Additional genes related to these genes and/or related to efficiency of the “fork reversion and repair” pathway have been identified including but not limited to WRN. BRCA1, ZRANB3, CDC6, CDT1, POLH, POLI, FANCD2, INO80, FANCB, ASH2L, FAM35A, XRCC3, BRIP1, and RNF168. For example, many of these genes interact and/or work in conjunction with one of the differentially expressed genes including but not limited to: RNF168 interacts/works in conjunction with 53BP1 and RNF8; BRCA1 interacts/works in conjunction with BRCA2; BRIP1 interacts/works in conjunction with BRCA2; ZRANB3 interacts/works in conjunction with SMARCAL1; WRN interacts/works in conjunction with BLM; and XRCC3 interacts/works in conjunction with RAD51. Some of these genes work upstream or downstream from one of the differentially expressed genes, for example, FAM35A works downstream of 53BP1. Other of these genes work to improve the efficiency of the “fork reversion and repair” pathway. For example, FANCD2 resolves unreplicated DNA in mitosis, and CDC6 and CDT1 are involved in replication origin initiation.

In parallel to the elevated frequency of chromosomal abnormalities in human embryos, mutation rates in the human germ line are also greater than in mice. Based on the frequency of mosaic mutations in adult tissues, it is known that early embryonic cell cycles are mutagenic, with approximately 3-4 mutations per division occurring in the first cell cycle, about three times more than in differentiated cells. Disease-causing de novo mutations are estimated to occur in 1 in 213 to 1 in 448 births. Increasing the efficiency of human development by increasing genome stability may also reduce the number of disease-causing de novo mutations, and therefore increase the safety of human reproductive treatments.

Disclosed herein are methods of reducing or decreasing replication abnormalities and/or aneuploidies in an embryo as well as increasing genome stability and/or developmental potential of an embryo.

Disclosed herein are methods of reducing or decreasing replication abnormalities and/or aneuploidies in an embryo as well as increasing genome stability and/or developmental potential of an embryo comprising activating one or more kinases and/or their signaling pathway including but not limited to ataxia telangiectasia and Rad3-related protein (ATR), WEE1, and checkpoint kinase 1 (CHK1) in an oocyte. These methods can be accomplished by introducing the polynucleotide encoding the kinase or the kinase itself into an oocyte as well as variants, mutants, fragments, derivatives, homologues, or paralogues thereof. The method can also be accomplished by introducing into an oocyte a polynucleotide or polypeptide which activates the kinase and/or their signaling pathway, as well as variants, mutants, fragments, derivatives, homologues, or paralogues thereof. For example, ETAA1 and TOPBP1, both in their native and truncated forms, activate ATR. These methods can also be accomplished by introducing an agent into an oocyte which activates the kinase, such as a small molecule or other pharmacological agents.

Disclosed herein are methods of reducing or decreasing replication abnormalities and/or aneuploidies in an embryo as well as increasing genome stability and/or developmental potential of an embryo introducing into an oocyte a polynucleotide, polypeptide and/or agent which increases efficiency of the “fork reversion and repair” pathway and/or decreases detrimental outcomes such as fork collapse, translesion synthesis and/or gap formation.

Also disclosed herein are methods of reducing or decreasing replication abnormalities and/or aneuploidies in an embryo as well as increasing genome stability and/or developmental potential of an embryo using one or more polynucleotides and/or polypeptides including CHEK1 (CHK1), WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof.

Also disclosed herein is a method of reducing or decreasing replication abnormalities and/or aneuploidies in an embryo comprising introducing one or more nucleic acids or polynucleotides selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof, into an oocyte.

Also disclosed herein is a method of increasing genome stability and/or developmental potential of a human embryo comprising introducing one or more nucleic acids or polynucleotides selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof, into an oocyte.

Also disclosed herein is a method of reducing or decreasing replication abnormalities and/or aneuploidies in an embryo comprising introducing one or more polypeptides selected from the group consisting of CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof, into an oocyte.

Disclosed herein is a method of increasing genome stability and/or developmental potential of an embryo comprising introducing one or more polypeptides selected from the group consisting of CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof, into an oocyte.

Also disclosed herein is a method of reducing or decreasing replication abnormalities and/or aneuploidies in an embryo comprising introducing one or more agents which activate and/or increase the expression of one or more genes selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6. CDT1, POLH, POLI, FANCD2, INO80, FANCB, ASH2L, FAM35A, XRCC3, BRIP1, RNF168, and combinations thereof into an oocyte.

Further disclosed herein is a method of increasing genome stability and/or developmental potential of an embryo comprising introducing one or more agents which activate and/or increase the expression of one or more genes selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof into an oocyte.

In some embodiments, the embryo and the oocyte are human. In some embodiments, the embryo and the oocyte are non-human.

In some embodiments, the nucleic acid or polynucleotide is mRNA. In some embodiments, the nucleic acid or polynucleotide is DNA.

In some embodiments, one polynucleotide or polypeptide is introduced. In some embodiments, more than one polynucleotide or polypeptide is introduced. In some embodiments, two or more polynucleotides or polypeptides are introduced. In some embodiments, five or more polynucleotides or polypeptides are introduced.

In some embodiments, a biological equivalent of the one or more polynucleotides or polypeptides is introduced.

In some embodiments, the polynucleotide or polypeptide is introduced into the oocyte at the time of fertilization by the sperm, e.g., by injection.

In one embodiment, the polynucleotide is mRNA. The sperm is passed through a droplet containing the mRNA at a specified concentration, e.g., 100 ng/ul

This approach is very practical as it does not involve any additional workload to the IVF clinic.

An alternative is injection after fertilization of the oocyte by the sperm.

In some embodiments, the method is carried out during an in vitro fertilization procedure.

Polynucleotides or nucleic acids which can be used in the present methods include: those that encode ATR, CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, REV3L, RECQL, RIF1, RNF8, SETD1A, SHPRH, SMARCAL1, TDRD3, TOPBP1, TP53BP1, WRNIP1, XRCC2, WRN, BRCA1, ZRANB3, CDC6. CDT1, POLH, POLI, FANCD2, INO80, FANCB, ASH2L, FAM35A, XRCC3, BRIP1, and RNF168 protein; the entire ATR, CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, and RNF168 gene; a polynucleotide or nucleic acid that is substantially homologous to ATR, CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDN1, POLH, POLI, FANCD2, INO80, FANCB, ASH2L, FAM35A, XRCC3, BRIP1, and RNF168 gene; and a variant, mutant, fragment, homologue, paralogue, or derivative of the ATR, CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, and XRCC2 WRN, BRCA1, ZRANB3, CDC6, CDT1, POLH, POLI, FANCD2, INO80, FANCB, ASH2L, FAM35A, XRCC3, BRIP1, and RNF168 gene that produces a protein that maintains or increases its function.

It is noted that as used herein CHEK1 (CHK1), WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, and RNF168, as well as ATR can refer to the gene or the protein encoded for by the gene, as appropriate in the specific context utilized and is meant to include the DNA, RNA, mRNA, cDNA, recombinant DNA or RNA.

The human gene information found in GenBank for any of the listed genes or others that can used in the disclosed methods can be used to obtain polynucleotide or nucleic acid sequence.

In some embodiments, the nucleic acid or polynucleotide encodes one of the proteins listed herein. The encoded protein may have an amino acid sequence that is at least 85% identical to the amino acid sequence of human protein (e.g., an amino acid sequence that is 85%, 86%, 87%, 88%, 89%, 90%, 91%, 92%, 93%, 94%, 95%, 96%, 97%, 98%, 99%, or 100% identical to the amino acid sequence of human protein). In some embodiments, the encoded protein has an amino acid sequence that is at least 90% identical to the amino acid sequence of human protein (e.g., an amino acid sequence that is 90%, 91%, 92%, 93%, 94%, 95%, 96%, 97%, 98%, 99%, or 100% identical to the amino acid sequence of human protein). In some embodiments, the encoded protein has an amino acid sequence that is at least 95% identical to the amino acid sequence of human protein (e.g., an amino acid sequence that is 95%, 96%, 97%, 98%, 99%, or 100% identical to the amino acid sequence of human protein).

In some embodiments, the encoded protein has an amino acid sequence that differs from human protein by way of one or more amino acid substitutions, insertions, and/or deletions, such as by from 1 to 10, 1 to 15, 1 to 20, 1 to 25, or more, amino acid substitutions, insertions, and/or deletions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions). In some embodiments, the encoded protein has an amino acid sequence that differs from human protein by way of one or more conservative amino acid substitutions, such as by from 1 to 10, 1 to 15, 1 to 20, 1 to 25, or more, conservative amino acid substitutions (e.g., by 1, 2, 3, 4, 5, 6, 7, 8, 9, 10, 11, 12, 13, 14, 15, 16, 17, 18, 19, 20, 21, 22, 23, 24, 25, or more, conservative amino acid substitutions).

In some embodiments, the polynucleotide or nucleic acid encoding the protein has a nucleic acid sequence that is at least 70% identical to the nucleic acid sequence of human protein (e.g., a nucleic acid sequence that is 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the nucleic acid sequence of human protein). In some embodiments, the polynucleotide or nucleic acid encoding protein has a nucleic acid sequence that is at least 85% identical to the nucleic acid sequence of human protein (e.g., a nucleic acid sequence that is 85%, 86%, 87%, 88%, 89%, 90%, 91%, 92%, 93%, 94%, 95%, 96%, 97%, 98%, 99%, or 100% identical to the nucleic acid sequence of human protein). In some embodiments, the polynucleotide or nucleic acid encoding protein has a nucleic acid sequence that is at least 90% identical to the nucleic acid sequence of human protein (e.g., a nucleic acid sequence that is 90%, 91%, 92%, 93%, 94%, 95%, 96%, 97%, 98%, 99%, or 100% identical to the nucleic acid sequence of human protein). In some embodiments, the polynucleotide or nucleic acid encoding the protein has a nucleic acid sequence that is at least 95% identical to the nucleic acid sequence of human protein (e.g., a nucleic acid sequence that is 95%, 96%, 97%, 98%, 99%, or 100% identical to the nucleic acid sequence of human protein).

In some embodiments, the present disclosure the polynucleotide or nucleic acid encoding the protein is in the form of a viral vector, comprising the polynucleotide or nucleic acid encoding the protein.

Any suitable viral system could be utilized including AAV, lentiviral vectors, or other suitable vectors.

In some embodiments, the polynucleotide or nucleic acid is RNA. The RNA molecule can be transcribed in vitro. Alternatively, the RNA molecule can be chemically synthesized.

In other embodiments, the RNA can be introduced into the oocyte as a DNA molecule. In such cases, the DNA encoding the RNA can be operably linked to promoter control sequence for expression of the RNA in the cell or embryo of interest. For example, the RNA coding sequence can be operably linked to a promoter sequence that is recognized by RNA polymerase III (Pol III). Examples of suitable Pol III promoters include, but are not limited to, mammalian U6 or H1 promoters. In some embodiments, the RNA coding sequence is linked to a human U6 promoter. In other exemplary embodiments, the RNA coding sequence is linked to a human H1 promoter.

The DNA molecule encoding the RNA can be linear or circular. In some embodiments, the DNA sequence encoding the RNA can be part of a vector. Suitable vectors include plasmid vectors, phagemids, cosmids, artificial/mini-chromosomes, transposons, and viral vectors. In some embodiments, the DNA encoding the RNA is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some embodiments, the DNA molecules encoding different RNAs are part of separate molecules (e.g., different vectors). In some embodiments, the DNA molecules encoding the different RNAs are part of the same molecule (e.g., same vector).

The polynucleotides or nucleic acids can be introduced into an oocyte by a variety of means. In some embodiments, the oocyte is transfected. One suitable method is electroporation. Other suitable transfection methods include calcium phosphate-mediated transfection, nucleofection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y, 3.sup.rd edition, 2001).

In other embodiments, the molecules are introduced into the oocyte by microinjection.

Alternatively, administering the proteins CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2 WRN, BRCA1, ZRANB3, CDC6, CDT1, POLH, POLI, FANCD2, INO80, FANCB, ASH2L, FAM35A, XRCC3, BRIP1, and RNF168 can be used in the methods. This includes the administration of a polypeptide or polypeptides, or a variant, mutant, fragment, homologue, or paralogue thereof having at least 80% sequence identity with any given polypeptide.

In an embodiment, the variant of the polypeptide has at least 81% sequence identity with the sequence of the polypeptide of which it is a variant. Thus, preferably, the variant of the polypeptide has at least 81%, 82%, 83%, 84%, 85%, 86%, 87%, 88%, 89%, 90%, 91%, 92%, 93%, 94%, 95%, 96%, 97%, 98% or 99% sequence identity with the sequence of any of the listed polypeptides. Such variants may be made, for example, using the methods of recombinant DNA technology, protein engineering and site-directed mutagenesis, which are well known in the art, and discussed in more detail below.

The percent sequence identity between two polypeptides may be determined using suitable computer programs.

Biologically active fragments (also referred to as biologically active peptides) or variants include any fragments or variants of a protein that retain an activity of the protein.

Polypeptides may be prepared using an in vivo or in vitro expression system. Preferably, an expression system is used that provides the polypeptides in a form that is suitable for pharmaceutical use, and such expression systems are known to the skilled person. As is clear to the skilled person, polypeptides of the invention suitable for pharmaceutical use can be prepared using techniques for peptide synthesis.

The polypeptide or variants or fragments thereof, may be made by chemical synthesis, again using methods well known in the art for many years. In certain embodiments, polypeptides for administration to a patient may be in the form of a fusion molecule in which the polypeptide is attached to a fusion partner to form a fusion protein. Many different types of fusion partners are known in the art. One skilled in the art can select a suitable fusion partner according to the intended use of the fusion protein. Examples of fusion partners include polymers, polypeptides, lipophilic moieties, and succinyl groups. Certain useful protein fusion partners include serum albumin and an antibody Fc domain, and certain useful polymer fusion partners include, but are not limited to, polyethylene glycol, including polyethylene glycols having branched and/or linear chains. In certain embodiments, the polypeptide may be PEGylated, or may comprise a fusion protein with an Fc fragment.

In an embodiment, the polypeptide may be fused to or may comprise additional amino acids in a sequence that facilitates entry into cells (i.e., a cell-penetrating peptide). Thus, for example, the protein or variant thereof or a polypeptide may further comprise the sequence of a cell-penetrating peptide (also known as a protein transduction domain) that facilitates entry into cells. As is well known in the art, cell-penetrating peptides are generally short peptides of up to 30 residues having a net positive charge and act in a receptor-independent and energy-independent manner.

It is appreciated that the sequence of the cell-penetrating peptide may be adjacent to the sequence of the protein or variant, or these sequences may be separated by one or more amino acids residues, such as glycine residues, acting as a spacer.

Other protein modifications to stabilize a polypeptide, for example to prevent degradation, as are well known in the art may also be employed. Specific amino acids may be modified to reduce cleavage of the polypeptide in vivo. Typically, N- or C-terminal regions are modified to reduce protease activity on the polypeptide. A stabilizing modification is any modification capable of stabilizing a protein, enhancing the in vitro half-life of a protein, enhancing circulatory half-life of a protein and/or reducing proteolytic degradation of a protein. For example, polypeptides may be linked to the serum albumin or a derivative of albumin. Methods for linking polypeptides to albumin or albumin derivatives are well known in the art.

The fusion partner may be attached, either covalently or non-covalently, to the amino-terminus or the carboxy-terminus of the polypeptide. The attachment may also occur at a location within the polypeptide other than the amino-terminus or the carboxy-terminus, for example, through an amino acid side chain (such as, for example, the side chain of cysteine, lysine, histidine, serine, or threonine).

In some embodiments, the one or more polynucleotide or polypeptide is introduced into an oocyte at the same time as a sperm cell. This can be accomplished using intracytoplasmic sperm injection (ICSI). In some embodiments, the one or more polynucleotide is mRNA. In some embodiments, the mRNA is synthetic.

In some embodiments, an agent is used. Such agents include but are not limited to small molecules and other pharmacological agents.

The method further comprises maintaining the embryo under appropriate conditions such that karyotyping can be performed. An embryo can be cultured in vitro (e.g., in cell culture). Typically, the embryo is cultured at an appropriate temperature and in appropriate media with the necessary O₂/CO₂ ratio to allow the expression of the RNA, if necessary. Suitable non-limiting examples of media include Global Total, M2, M16, KSOM, BMOC, and HTF media. A skilled artisan will appreciate that culture conditions can and will vary.

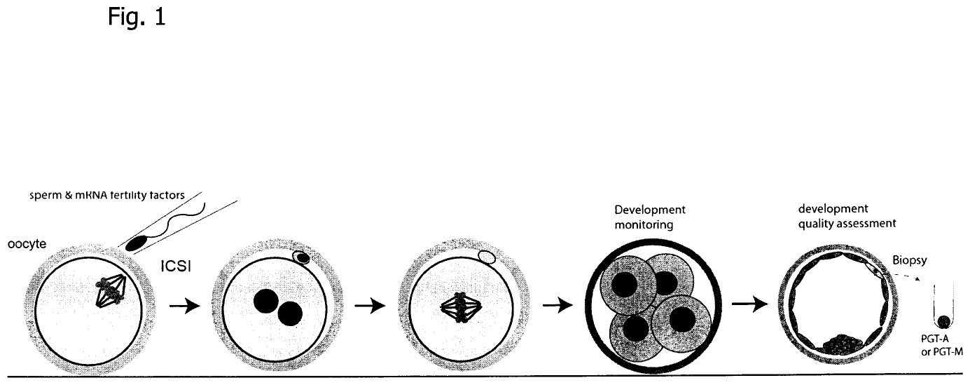

The embryo is then cultured to the blastocyst stage and a tropectoderm biopsy is performed to determine the karyotype. Embryos with a normal genotype are implanted. The blastocyst may be frozen prior to implantation. See FIG. 1.

The present disclosure also provides kits comprising the reagents for practicing any of the methods disclosed herein.

In one embodiment, the kit includes one or more polynucleotides or polypeptides that activate one or more kinases including but not limited to ATR, WEE1, and CHK1.

In a further embodiment, the kit includes one or more agents which activate one or more kinases including but not limited to ATR, WEE1, and CHK1.

In one embodiment, the kit includes one or more polynucleotides or polypeptides selected from the group consisting of CHEK1 (CHK1), WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6. CDT1, POLH, POLI, FANCD2, INO80, FANCB, ASH2L, FAM35A, XRCC3, BRIP1, RNF168, variants, mutants, fragments, derivatives, homologues, or paralogues thereof and/or delivery systems of such polynucleotides or polypeptides such as vectors.

In one embodiment, the kit includes one or more agents which activate and/or increase the expression of one or more genes selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RB1, RECQL, REV3L, RIF1, RNF8, SETD1A, SHPRH, SMARCAL1, TDRD3, TOPBP1, TP53BP1, WRNIP1, XRCC2, WRN. BRCA1, ZRANB3, CDC6, CDT1, POLH, POLI, FANCD2, INO80, FANCB, ASH2L, FAM35A, XRCC3, BRIP1, and RNF168.

In some embodiments, the kit includes an mRNA of the desired factor in a formulation that can be combined with sperm to be injected into an oocyte at the time of an in vitro fertilization.

In some embodiments, the kit may include additional reagents such as those for culturing the embryos and testing the embryos as well as instructions.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Methods and Materials for Examples 2-11

### Human Subjects

The Columbia University Institutional Review Board, the Columbia University Embryonic Stem Cell Committee approved all human procedures to obtain human oocytes for this research, including for low pass genome sequencing and public database submission. Gamete and embryo donors provided informed consent, and followed guidance provided by New York State Stem Cell Science. The experience of oocyte donors in research is described in a prior study (Zakarin Safier et al., 2018).

### Human Oocytes

Human oocytes designated for research were obtained from donors following routine gonadotropin releasing hormone antagonist in vitro fertilization stimulation at the Columbia University Fertility Center. Ovarian stimulation was monitored with regular ultrasound and serum hormonal assays per practice protocol (Palmerola et al., 2018). Oocyte retrieval was performed by transvaginal ultrasound-guided needle aspiration under intravenous sedation. Oocytes were vitrified using Cryotech vitrification kit and stored in liquid nitrogen until use. For experimental procedures, oocytes were thawed using the Cryotech Warming Kit (Cryotech, Tokyo, Japan) or the ECMP warming solution set, then exposed to experimental conditions as outlined below. For the ensuing human oocyte studies, “control media” refers to Global Total. All experiments were conducted during incubation at 37° C. with 5% CO₂ and 20% O₂. Donated oocytes were used for experiments involving artificial activation and for experiments involving timing of DNA replication in zygotes, Cas9 injection, as well as for ICSI followed by aphidicolin incubation. Human oocytes were randomly allocated to experimental groups.

### Human Embryos

Cryopreserved human cleavage-stage and blastocyst embryos and unfertilized oocytes were anonymously donated from individuals and couples who provided consent for use in research. Embryos were of good quality and had been cryopreserved for potential clinical use but were then no longer needed and donated for research. The cause of treatment is not disclosed. Range of maternal age of donated embryos is between 27-43. Embryos of both sexes were used, as provided by the unbiased fertilization of oocytes with sperm carrying either a Y or X chromosome. Cleavage stage embryos were thawed using the one-step thaw protocol using Global total with HEPES. Blastocyst stage embryos were thawed using the Quinn's Advantage thaw kit, then exposed to experimental conditions as outlined below. Vitrified embryos were thawed with the ECMPC warming solution set. Embryo culture was performed in Global Total. All experiments were conducted during incubation at 37° C. with 5% CO₂ and 20% 02.

### Sperm Donation and Processing

Human sperm was obtained following donation from living donors following masturbation after 2-5 days of abstinence and collected in a Male-FactorPac collection kit. Sperm was cryopreserved using commercially prepared density gradient (Isolate) and centrifugation was used to isolate motile sperm. The resultant sperm pellet was resuspended in sperm prep medium (Quinn's Sperm Washing Medium, Origio), centrifuged and washed repeatedly prior to cryopreservation using freezing medium (Irvine Scientific, 90128). Cryopreservation was performed using a time-based freezing protocol with gradually lowered temperature time sets then stored in liquid nitrogen at less than −196° C. until use. Samples were thawed at 38-40° C. for a maximum of 10 min and transferred to a 15 mL conical tube. Quinn's Sperm Washing Medium was added dropwise to achieve a volume of 6 mL. Samples were centrifuged at 300×g for 15 min. Supernatant was removed and an additional wash with centrifugation was performed. The supernatant was removed following a second wash, sperm pellets resuspended in wash media, then analyzed for viability. Sperm was mixed on the stage of a microscope with 10% PVP, and individual motile sperm were immobilized by pressing the sperm tail with the ICSI micropipette to the bottom of the dish, then picked up for injection. Donated sperm was used to determine the timing of DNA replication in zygotes, as well as for ICSI followed by aphidicolin incubation.

### Murine Oocytes

The Columbia Institutional Animal Care and Use Committee approved animal protocols. Mouse oocytes were obtained as described previously (Yamada and Egli, 2017). Briefly, B6D2F1 female mice (Jackson strain #100006) were superovulated at 5-7 weeks of age by injection with 5 IU of pregnant mare serum gonadotropin (ProSpec) followed 48-52 h later by 5 IU of human chorionic gonadotropin (EMD Millipore). Mice were sacrificed 14-15 hours later and oviducts were removed. Oocyte-cumulus complexes were isolated from the oviduct in a dish containing droplets of GMOPSplus (Vitrolife) containing 300 mg/mL hyaluronidase (Sigma-Aldrich), covered with mineral oil on the heated stage of an Olympus SZ51 microscope. Oocytes were then washed and cultured in Global Total until experimental use.

### Embryo Culture and Drug Incubations

Embryos, mouse and human, were cultured in Global Total (Life-Global) for the duration of the experiments and with added compounds as outlined below. Incubation in RNA polymerase inhibitors triptolide (1 mM) or alpha amanitin (20 mg/ml) was performed from the 2PN stage (day1) to day3 of human development. Incubation in aphidicolin was performed at 2 mM. This concentration of aphidicolin has been shown to prevent the formation of ssDNA gaps through uncoupling of leading and lagging strand synthesis (Ercilla et al., 2020). The following dosages were used: CHK1/CHK2 inhibitor AZD7762 (Zabludoff et al., 2008) at 100 nM, CHK1 inhibitor LY2603618 (King et al., 2014) at 2.5 mM, Wee1 kinase inhibitor MK1775 (Hirai et al., 2009) at 500 nM and ATR inhibitor ATR45 (VE-821), which shows selectivity of >600× over related kinases ATM or DNA-PK (Charrier et al., 2011), at 1 mM, except for zygotes at the 1-cell stage which were incubated for 4 hours at 2.5 mM ATR45. PFM01 (Shibata et al., 2011) was used at 50 mM, and human zygotes were incubated from 14-19 h post activation, while mouse zygotes were incubated from 11.5-15.5 h post activation in aphidicolin and either mirin or PFM01. Mirin was used at a concentration of 50 mM. Olaparib was used to inhibit PARP at a concentration of 10 mM. All experiments were conducted during incubation at 37° C. with 5% CO₂ and 19-20% O₂. Chemical stock solutions were prepared in DMSO at concentrations at least 1000× higher than the final concentration.

### Cas9 RNP Preparation and Injection

gRNA was obtained from Integrated DNA Technologies (IDT). For ribonucleoprotein (RNP) preparation, 2 mL of 63 mM IDT nlsCas9 v 3, 3 mL of injection buffer, and 1.5 mL of 100 mM sgRNA were mixed and kept at room temperature for 5 minutes. Thereafter, 96.5 mL of injection buffer consisting of 5 mM Tris-HCl, 0.1 mM EDTA, pH 7.8 was added prior to injection. gRNAtargeted chr6, chr11, chr16 and chr17. Injection was performed into metaphase MII oocytes.

### Artificial Activation

Artificial activation of human MII oocytes was performed using two pulses of 2.7 kV/cm at 50 ms using ECFG21 (Nepagene) in fusion buffer consisting of 0.25 M d-sorbitol buffer containing 0.1 mM calcium acetate, 0.5 mM magnesium acetate, 0.5 mM HEPES, and 1 mg ml⁻¹ fatty-acid-free BSA as previously described (Tachibana et al., 2013). Thereafter, oocytes were incubated for 3.5-4 h in 10 mg/ml puromycin, followed by a wash step and following culture in Global total.

Mouse oocytes were activated at 1-6 hours post retrieval using 2 mM ionomycin (Sigma Aldrich) in Global Total medium for 5 minutes at 37° C. followed by culture in 10 mM puromycin and 5 mg/ml cytochalasin B for 3 hours, then cytochalasinB only for 2 hours and 10 minutes to prevent second polar body extrusion.

### Immunocytochemistry of Human and Murine Embryos

Following the experimental exposures, mouse and human embryos were fixed in 2% paraformaldehyde (PFA) and 0.25% Triton X-100 in PBS for 10 minutes at room temperature, serially washed in phosphate-buffered solution (PBS) at room temperature, then stored in PBS at 4° C. until staining and imaging. Embryos were placed in 10-20 ml droplets of primary antibody solution within plastic tissue culture dishes covered with mineral oil for 1 hour at room temperature, serially washed (×2) in PBS containing 0.01% Triton X-100 to prevent embryos sticking to oil, then transferred to 10-20 ml secondary antibody solution and Hoechst (dilution 1:500) for 30 minutes. Embryos were then serially washed (×2) and placed in 5 ml droplets of PBS with 0.5% serum albumin in uncoated glass bottom dishes (Mattek) covered with embryo culture oil for imaging. The following primary antibodies were used across experiments as described above: Oct-4, γH2AX, RPA32, RPA32 S4/S8, RPA32 S33, 53BP1, RAD51, Centromere, and phospho-CHK1. Phospho-specific antibodies were quality controlled through ATR dependence of foci formation in mouse zygotes, and for γH2AX also in human zygotes. The RPA32 phospho S33 antibody does not react to an S33A mutant (Anantha et al., 2007). The Anti-phospho-Histone H2A.X-Ser139 mouse monoclonal antibody does not react to S139A in human pluripotent stem cells (Orlando et al., 2021). The RPA32 (S4/S8) rabbit antibody does not stain RPA32 mutant with serines turned to alanine (Zuazua-Villar et al., 2015; Liu et al., 2012). The appropriate secondary antibodies conjugated with Alexa Fluor 488, Alexa Fluor 555, CF 568, or Alexa Fluor 647 were diluted at 1:500-1:1000. Ethynyl deoxyuridine (EdU) labeling of zygotes and embryos was performed using the labeling kit (Thermo Fisher Scientific) as described by the manufacturer. 10 mM EdU was added to culture medium for 1 h at the indicated time points, and cells were harvested and fixed thereafter. 5-ethynyl uridine (EU) labeling was performed by incubating day3 cleavage stage embryos for 4 hours in Global total containing 0.2 mM EU.

### DNA Fiber Analysis

DNA fiber analysis was performed in mouse 1-cell embryos at 5.5 h post activation, in late G2 phase from 12-15 h post activation, in 8-cell embryos at 52 h post activation, and in blastocysts at 96 h post activation. Human 1-cell embryos were labeled 6 h post activation, and donated day3 and day6 embryos were labeled within 3-4 h after thawing. Incubation was performed in 25 mM IdU or 25 mM CldU for 30 min. Digestion of zona pellucida by Acidic Tyrode's solution was performed in a 4-well dish at room temperature at ambient atmosphere for 1-5 minutes using continuous observation under a stereomicroscope, and then neutralized in culture medium. Approximately 30 cells were collected in 0.5-1 ml medium and placed in a PCR tube, lysed by adding 30 ml of fresh pre-warmed (30° C.) spreading buffer (0.5% SDS, 200 mM Tris pH 7.4, 50 mM EDTA), incubated for 6 min at RT and stretched using gentle tilting on regular microscope slide. Slides were fixed for 2 min at RT in cold 3:1 methanol: acetic acid, air dried at RT, and then incubated in 2.5M HCl for 30 min, rinsed 5 times with PBS, and blocked with 3% BSA in PBS for 1 h. DNA fibers were stained for 1 h with anti-ssDNA and anti-BrdU/IdU antibodies. Afterwards, slides were rinsed 4 times in PBS, incubated 1 h with secondary antibodies (Goat anti-mouse IgG1, #A21121; Goat anti-mouse IgG2a, #20258), mounted with ProLong Gold Antifade mountant and let dry overnight. The fiber tracks were imaged on a Nikon Eclipse 90i microscope fitted with a PL Apo 40X/0.95 numerical aperture objective and measured using ImageJ software v 1.53.

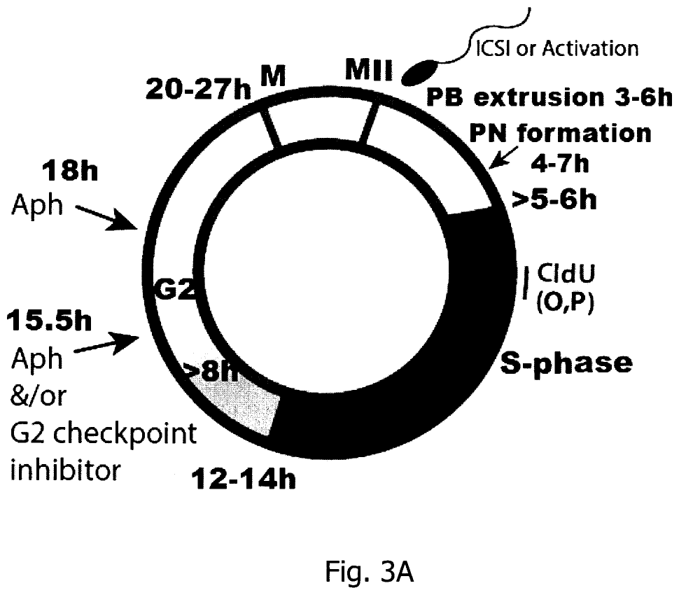

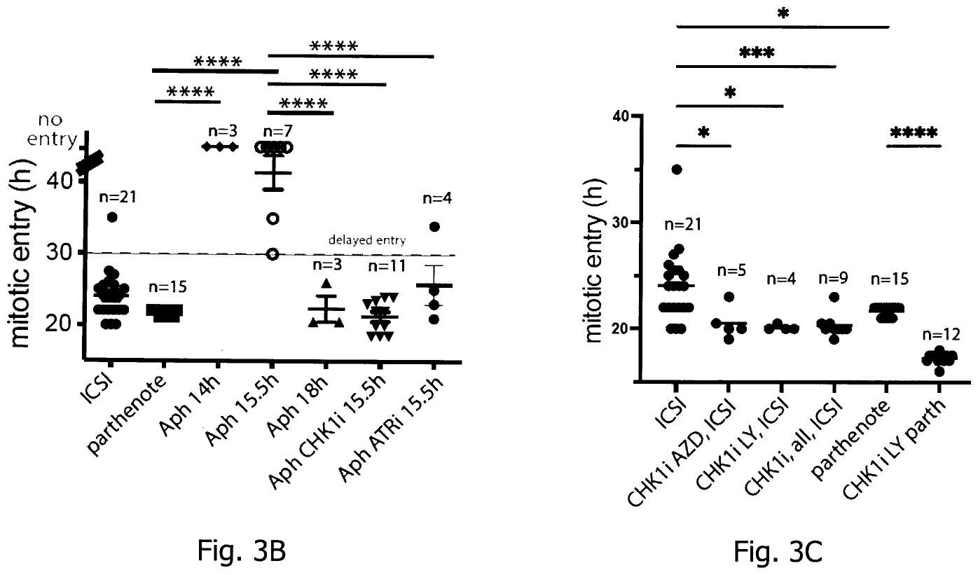

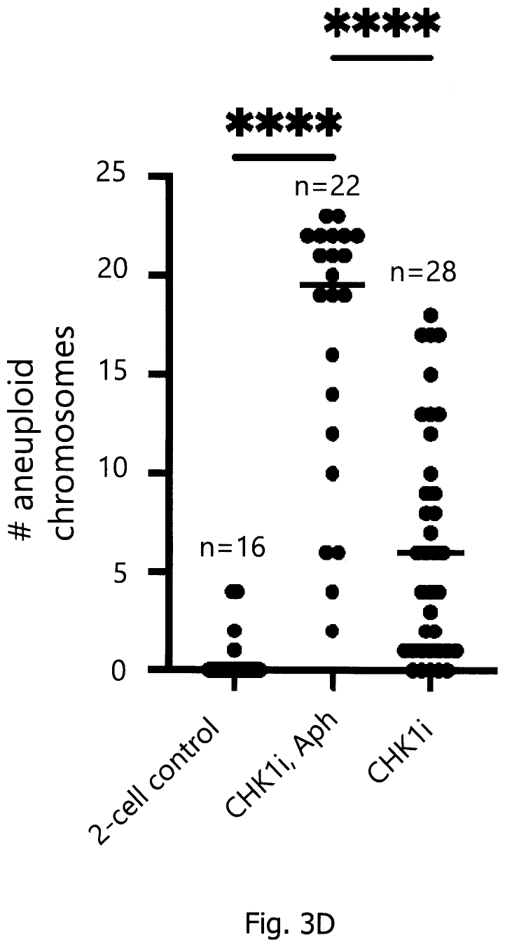

The length of each track was determined manually using the segmented line tool on ImageJ software. The pixel values were converted into mm using the scale bar generated by the microscope software. The extension of the DNA fibers was calculated as follows: 2.59±0.24 kbp/mm according to (Jackson and Pombo, 1998). In FIG. 3, sister fork ratio was calculated as shorter fork/longer fork, with bidirectional forks only. Forks were considered asymmetric with a ratio <0.75 as (Bester et al., 2011).

### Confocal Microscopy and DNA Damage Foci Analysis

Embryos were imaged with confocal microscopy using a Zeiss LSM (LSM 710) at a magnification of 63× and the Zeiss Zen software. Images were obtained using 488-, 543- and 633-nm lasers and bright-field microscopy. The number of confocal sections was adjusted to capture the entire nucleus or the entire embryo according to the experimental design as described above. Cell numbers and staining foci numbers were counted manually. Nuclei were identified by the presence of Hoechst33342. Foci were identified if at least 3× greater intensity than nuclear average, spherical in appearance, and overlapping with Hoechst33342 staining. Inner cell masses were identified by the presence of Oct4 within a blastocyst embryo. Active DNA replication was identified by the presence of EdU. DNA damage was identified by the detection of γH2AX and RPA foci within the nucleus. Mechanisms of DNA repair were identified through the detection of RPA S4/S8, RPA S33, Rad51 and 53BP1 foci within the nucleus.

### Single-Cell/Single Nuclei Whole Genome Amplification and Sequencing

Single cells from blastomeres were collected on the heated stage (Tokai Hit) of an Olympus IX71 inverted microscope equipped with Narishige micromanipulators and a zona pellucida laser (Hamilton-Thorne). Single nuclei were isolated from blastomeres by lysis and dissection of different nuclei using two 20 mm diameter Piezo micropipettes and Holding pipette (Origio). Single embryo cells or nuclei were placed manually in single wells of a 96-well plate containing 9 ml of lysis buffer, prepared as a master mix of 798 ml H₂O, 6 ml of 10 mg/mL Proteinase K solution (Sigma-Aldrich), and 96 ml 10× Single Cell Lysis and Fragmentation buffer (Sigma-Aldrich). Single cells/single nuclei were lysed by heating 96-well plates containing single cells/single nuclei at 50° C. for 1 hour, followed by incubation at 99° C. for four minutes using a PCR thermocycler. Single-cell/single-nuclei whole genome amplification (WGA) using degenerate oligo-nucleotide priming PCR (DOP-PCR) was performed using the SEQPLEX Enhanced DNA Amplification Kit (SEQXE, Sigma) according to the manufacturer's instructions. In addition, a modified version of the DOP-PCR protocol that allows inline indexing of WGA DNA was also applied. WGA DNA was subsequently processed for Illumina library sequencing preparation using standard TruSeq indexing methods via the NEBNext Ultra II DNA Library Prep Kit for Illumina (New England Biolabs). NEBNext Multiplex Oligos for Illumina (96 index primers) were used for four amplification cycles. Library DNA was quantified using Qubit dsDNA HS Assay Kit (Invitrogen, Carlsbad, CA, USA) and library size distribution was confirmed using a 2100 Bioanalyzer DNA 1000 Kit (Agilent, Santa Clara, CA, USA). A unimodal curve centered around 300-500 bp was scored as a successful library preparation. Subsequently, 30 mL of each pooled library was sent for sequencing at a concentration of 20 ng/ml. Mutiplex sequencing was performed using Illumina HiSeq 2500 single end indexed run (SR100) while targeting a coverage of approximately 1 million reads per cell/nuclei, sufficient for quantitative genome-wide copy number analysis.

### Single-Cell/Single-Nuclei Sequencing Analysis and Copy Number Inference

Analysis of DNA sequencing data was performed as previously described (Baslan et al., 2015) with minor modifications as outlined below. Briefly, multiplex sequencing data were demultiplexed according to unique barcodes with reads assigned to their respective single-cell or single-nuclei. Unique reads were aligned to the reference human and mouse genome builds, hg19 and mm9, respectively. Each genome was partitioned into 5000 bins using a previously described algorithm (Varbin) that corrects for genome sequence mappability (Baslan et al., 2012; Navin et al., 2011). Uniquely mapped reads were sorted, indexed and subsequently counted within the partitioned genomic bins. For single-cell copy number inference, read count data were normalized genome-wide followed by CBS segmentation (Olshen et al., 2004) and transformed into integer copy number states using a least-squares fit approach (Baslan et al., 2015).

Copy number inference from single-nuclei sequencing data was performed similarly with the exception that bin read counts were initially processed using Kernel Density Estimation (KDE) resulting in two primary densities. One density contained very low read counts and was attributed to index switching during Illumina multiplex library sequencing, a known sequencing artifact (Larsson et al., 2018). Bins constituting this density were normalized and transformed to a ground state (i.e., nullisomy). The second density contained high read counts in different genomic positions in one or more chromosomes across different single-nuclei. Read counts were processed similarly to single-cell data with the exception that segments were designated as “copy number states” (compared to absolute integer) given the inherent uncertainty of the absolute copy number of the segments in spontaneous micronuclei.

For increased resolution of break point mapping, additional analysis with bin size of 10 kb, 50 kb and 100 kb using bwa aligner (v. 0.7.17) (Li and Durbin, 2009), samtools (v. 1.11) (Danecek et al., 2021), and R package QDNAseq v 1.26.0 (Scheinin et al., 2014) was performed and results evaluated for consistency with analysis using 500 kb bins described above (Baslan et al., 2015). All break point calls were also manually inspected on copy number plots and read number files independently by two researchers. Parthenogenetic 1-cell embryos were haploid, enhancing signal differences, and allowing for increased resolution of chromosomal break mapping to 10-50 kb.

### Break Site Annotations

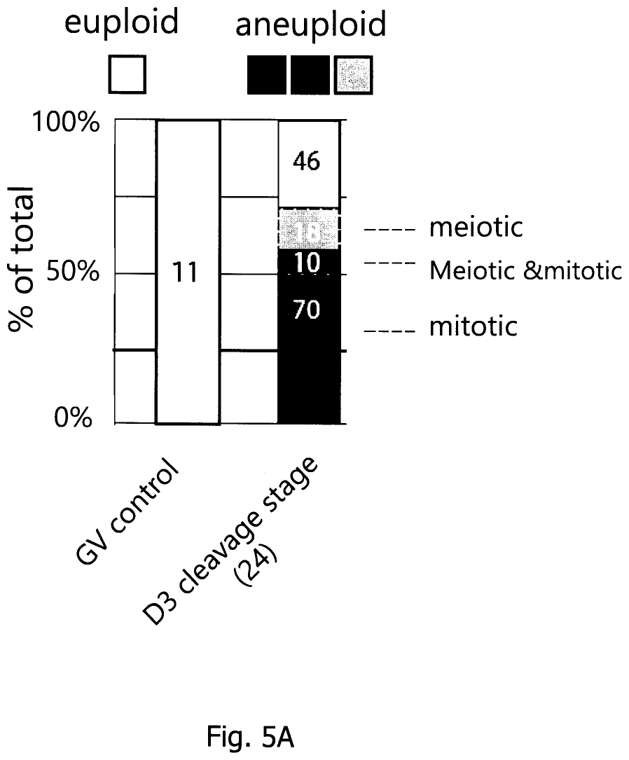

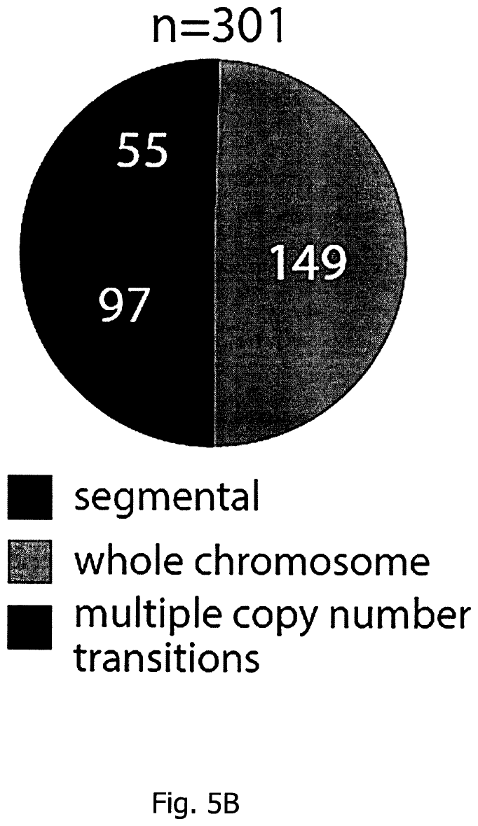

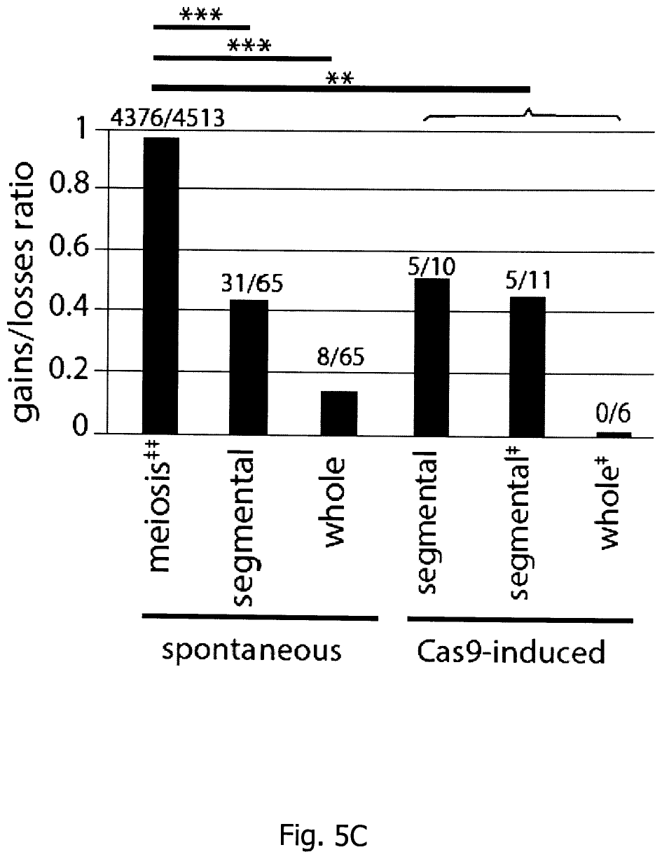

Annotation of break sites was performed as follows: transition points in segmented raw read count data were flagged across single-cell/single-nuclei datasets with subsequently manual curation using the University of California Santa Clara (UCSC) Genome Browser and transition point coordinates. A site was considered fragile if it occurred independently within the same or a neighboring bin (according to Baslan et al., 2015) in at least three different embryos. A fragile area is defined by the bin coordinates within which the breaks fall (as in FIG. 5 for CNTN5). Gene density comparison was performed by selecting random locations within hg19 genome assembly using bedtools v 2.30.0 (Quinlan and Hall, 2010). Complete transcripts and adjacent intergenic areas were included in 1-2 Mb intervals to calculate the density of protein-coding genes. The genome average density of protein coding genes of approximately 6.9 genes/Mb is based on an estimated 21,306 protein-coding genes (Pertea et al., 2018) and a human genome size of 3.1 billion base pairs.

### Statistical Analysis of DNA Damage and Embryo Development

Statistical analyses were performed on GraphPad Prism (GraphPad Software, Inc., La Jolla, CA, USA). Embryo groups were compared using one-way analysis of variance (ANOVA) with Bonferroni correction for multiple comparisons where appropriate and using Student's t-test for comparison between two groups. Fisher's exact test was performed using https://www.socscistatistics.com/to analyze sister fork asymmetry at different developmental stages, and to analyze development of human embryos with and without CHK1 inhibitor as indicated in Figure Legends. Mean and standard deviations are displayed as indicated in Figure Legends. The number of n indicated in the Figures represents the number of cells. In the case of zygotes, the number of embryos and the number of cells is equivalent.

### Example 2—Spontaneous DNA Damage Forms During the First Cell Cycle in Human Embryos

The first cell cycle in human progresses within a one day window from fertilization to mitosis.

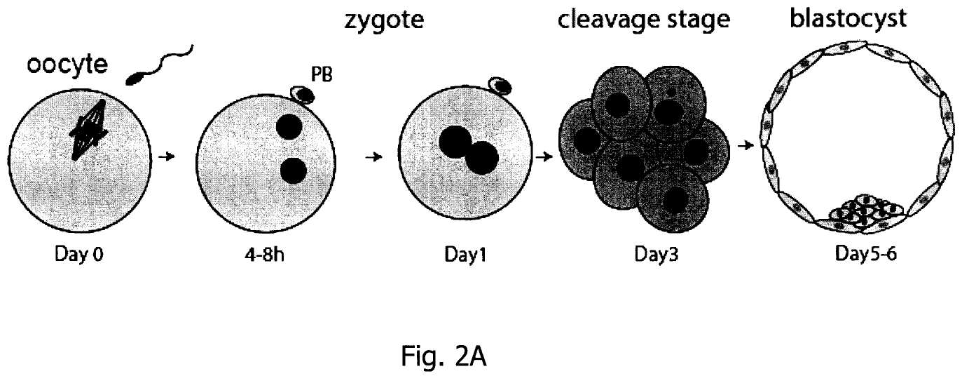

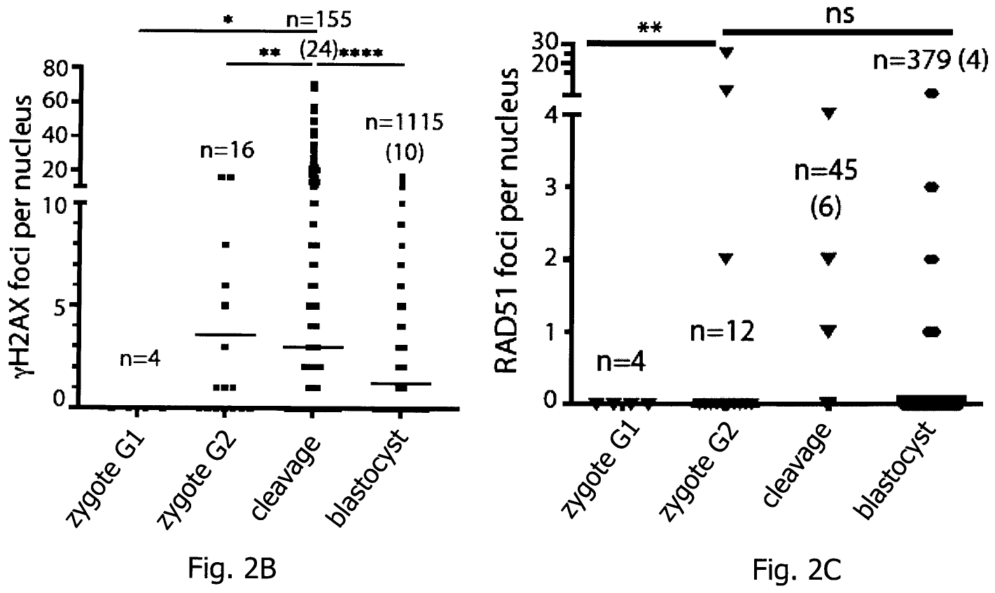

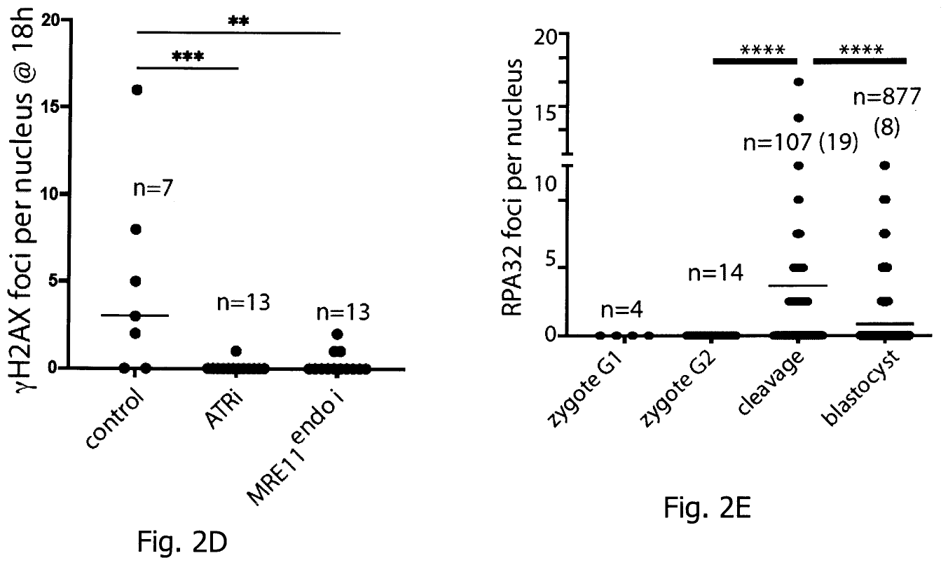

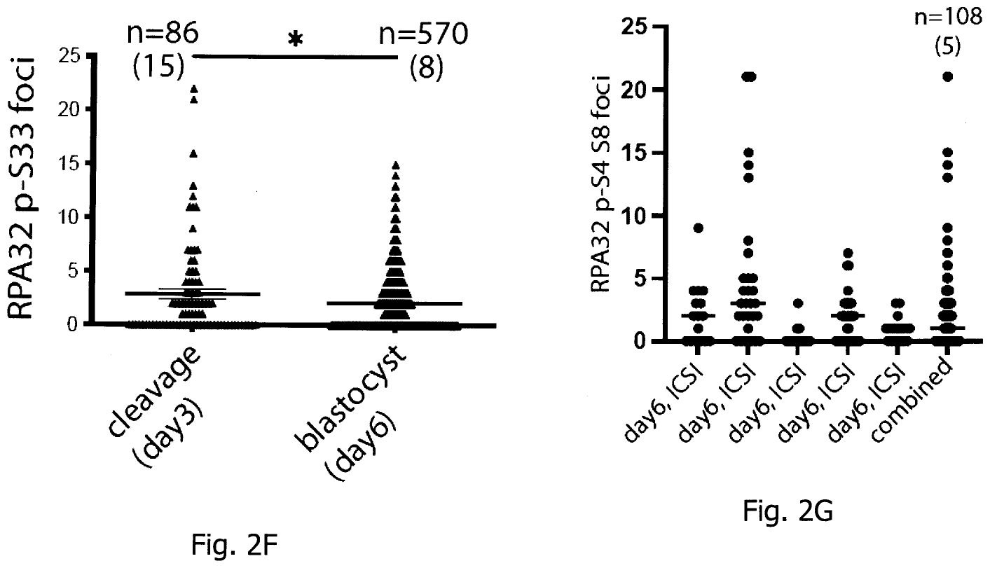

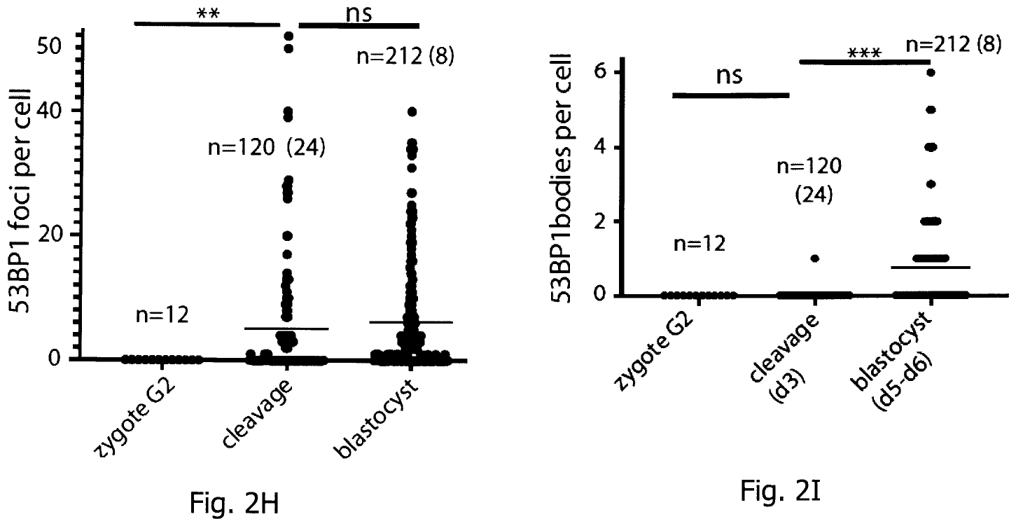

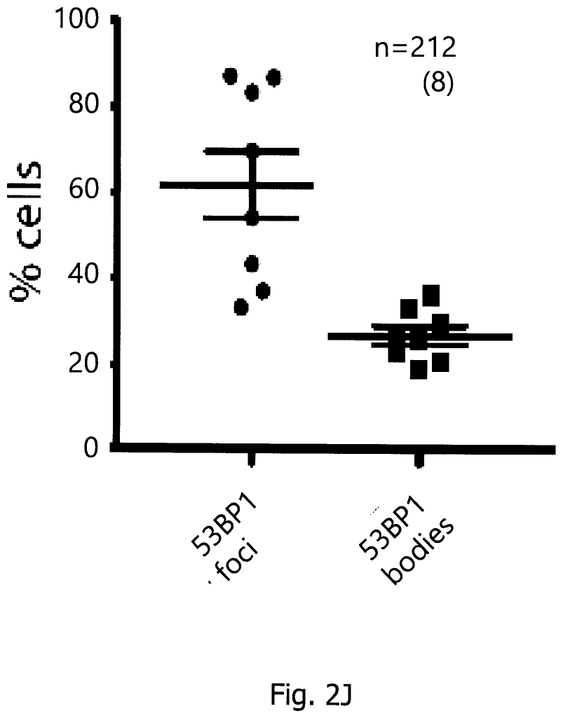

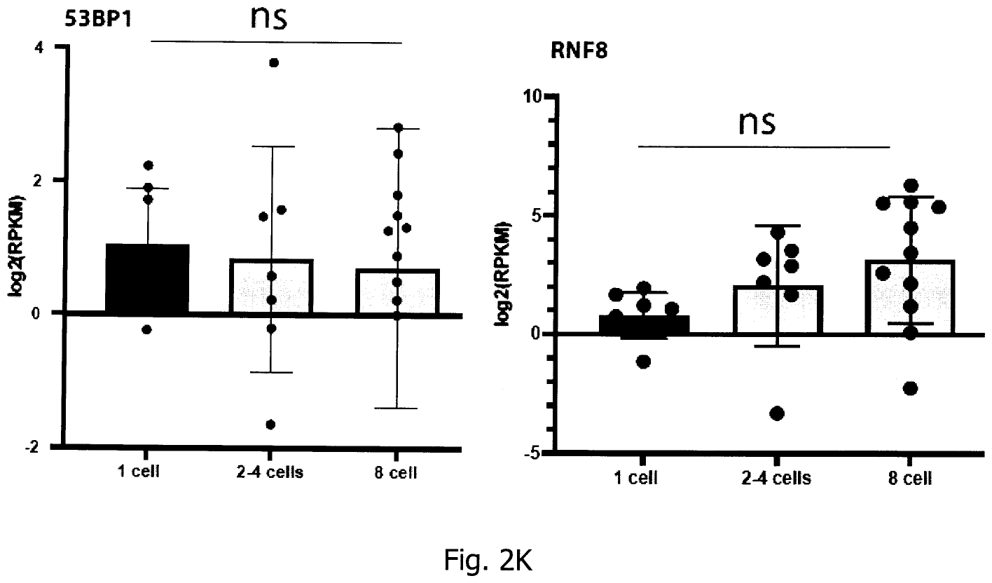

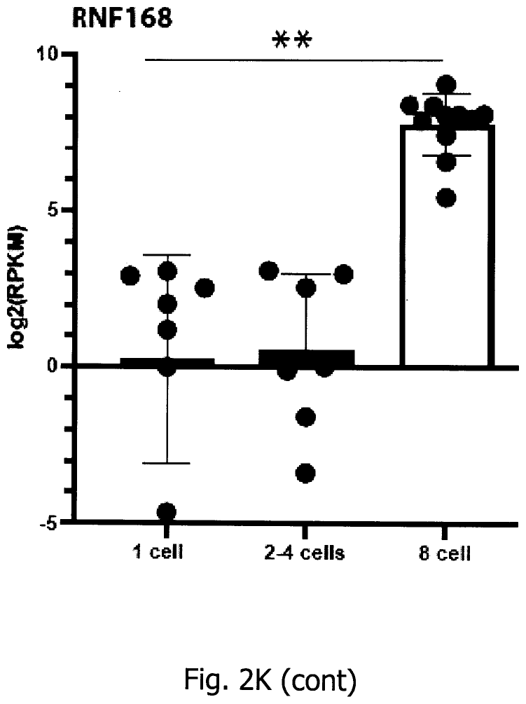

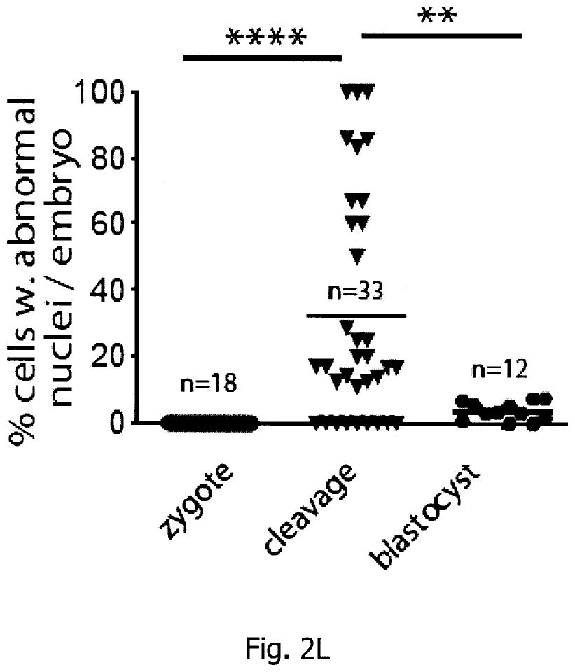

To distinguish DNA damage inherited from the gamete versus DNA damage acquired during cell-cycle progression, we stained zygotes on day 0, shortly after fertilization and pronucleus formation, as well as on day 1, shortly before the first mitosis (FIG. 2A). No foci for γH2AX or RAD51 were seen at 1-2 h post pronuclear appearance, but both γH2AX foci and RAD51 foci were found on day 1 and shortly before mitosis in human zygotes (FIG. 2B-2C). The phosphorylation of γH2AX at Ser139 can be mediated through the activation of ATR kinase, which is activated in response to DNA replication stress (Ward and Chen, 2001). When parthenogenetically activated oocytes were incubated in ATR (ataxia telangiectasia and Rad3-related protein) inhibitor from 14-18 h into the first cell cycle, the number of γH2AX foci was reduced significantly relative to controls evaluated at the same time point (FIG. 2D). Therefore, foci indicating DNA damage and repair form de novo after the first S phase. These foci may be DSBs at collapsed replication forks or ssDNA gaps that emerge at stalled replication forks or at lesions behind the fork (Petermann et al., 2010; Kuo and Yang, 2008; Piberger et al., 2020).

MRE11 binds to DSBs at stalled forks and at ssDNA gaps to mediate resection, the formation of ssDNA tracts, and the loading of RPA and Rad51 to initiate repair (Piberger et al., 2020; Shibata et al., 2014). We inhibited MRE11 endonuclease activity using PFM01 (Shibata et al., 2014). In the presence of PFM01, the formation of γH2AX and Rad51 foci was inhibited (FIG. 2D). Thus, γH2AX and Rad51 foci in G2 phase zygotes represent DNA breaks or gaps that undergo MRE11-dependent processing and activate ATR signaling.

We also stained cleavage stage and blastocyst stages human embryos (FIG. 2A). Foci for γH2AX increased significantly at the cleavage stage (FIG. 2B), whereas RAD51 foci remained constant (FIG. 2C). Foci for RPA32, indicating extensive ssDNA (Raderschall et al., 1999), were first observed at the cleavage stage and decreased at the blastocyst stage (FIG. 2E). Consistent with ATR activation, RPA32 was found phosphorylated at serine S33 (FIG. 2F), as well as at S4 and S8 (FIG. 2G). RPA32 S33 phosphorylation is conferred by ATR (Vassin et al., 2009), whereas S4 and S8 are phosphorylated at DSBs by DNA-PK or ATM in dependence on S33 phosphorylation (Anantha et al., 2007; Liu et al., 2012; Liaw et al., 2011). Phosphorylation of RPA32 S33 was found in embryos fertilized by either intracytoplasmic sperm injection (ICSI) or by in vitro fertilization and was particularly notable in binucleated cells and in micronuclei. Furthermore, the phosphorylation of CHK1 at serine 317, a checkpoint kinase phosphorylated by and acting downstream of ATR (Liu et al., 2000), was also observed (results not shown). 53BP1 foci smaller than 1 mm that mark DSBs (Schultz et al., 2000) were absent on days 1 and 2 of development and increased significantly on day 3 (FIG. 2H). Interestingly, 53BP1 bodies larger than 1 mm in diameter were most common in blastocyst-stage embryos (FIG. 2I), present in approximately 20% of cells (FIG. 2J). The 53BP1 bodies indicate sites of repair of incompletely replicated DNA from the previous S phase passed on to daughter cells (Lukas et al., 2011; Harrigan et al., 2011), which is apparent in the symmetry of 53BP1 bodies and the presence of RPA32 marking ssDNA. The developmental timing of 53BP1 foci formation may be due to the absence of detectable RNF168 transcripts prior to embryonic genome activation (EGA) (FIG. 2K); RNF168 is required for the formation of 53BP1 foci at DSBs (Bohgaki et al., 2011; Doil et al., 2009).

Abnormal nucleation, defined by the presence of multiple nuclei within a single blastomere, paralleled the formation of RPA32 foci; although no abnormal nucleation was observed at the zygote stage, it was most common on day 3 at the cleavage stage and decreased at the blastocyst stage (FIG. 2L). Chromatin bridges, indicative of unresolved replication intermediates at mitosis, were also observed in cleavage-stage and blastocyst-stage embryos.

Thus, DNA damage is acquired de novo during the first cell cycle and at subsequent cleavage divisions. The formation of ssDNA, micronucleation, chromatin bridges, Rad51 foci, 53BP1 foci, and 53BP1 bodies and the phosphorylation of H2AX, RPA32, and CHK1 in preimplantation development are consistent with DNA replication stress, DSBs or a combination of breaks and ssDNA gaps, and with late or incomplete replication at entry into mitosis.

### Example 3—CHK1 Delays Mitotic Entry in Response to Unreplicated DNA in G2-Phase Human Zygotes

Spontaneous DNA damage can arise during S phase and thus we aimed to understand S-phase progression in human zygotes.

We incubated human zygotes with EdU in hourly intervals after fertilization beginning after pronucleus formation. Pronucleus (PN) formation occurred between 4 and 7.5 h post ICSI. EdU staining was noted from 6 to 13 h post ICSI but not after 14 h or more. Therefore, S phase is 6-7 h in human zygotes (FIG. 3A).

Mitotic entry occurred between 20 and 27 h post ICSI in 19 of 20 zygotes with an average of 24 h (FIG. 3B). Human parthenotes (n=22) were generated by an activation pulse. Pronuclei formed at 4 h post activation, and all human parthenotes (15/15) were in mitosis by 22 h (FIG. 3B). Thus, the longest phase of the cell cycle is G2, lasting 8-14 h (FIG. 3A). DNA damage foci observed on day 1 in human zygotes are in G2 phase cells, suggesting a continued requirement for DNA synthesis and/or repair.

In addition to EdU staining, we determined the timing of DNA replication completion by applying aphidicolin and monitoring mitotic progression. Aphidicolin introduces replication fork stalling by reversibly inhibiting B-type family DNA polymerases (Ikegami et al., 1978; Wright et al., 1994). Oocytes were activated by parthenogenesis, which allows the synchronous progression through the first cell cycle. Incubation with aphidicolin at 14 h prevented mitotic entry, whereas incubation at 15.5 h allowed mitotic entry with a delay compared with controls (FIG. 3B). Incubation with aphidicolin at 18 h had no effects on mitotic entry timing. In the presence of aphidicolin from 15.5 h post activation, foci for γH2AX and RPA32 formed within 6 h (results not shown). The ssDNA bound by RPA32 at remodeled forks activates an ATR-dependent checkpoint (Zou and Elledge, 2003). In the presence of an inhibitor of ATR kinase or of CHK1 kinase from 15.5 h post activation, zygotes entered mitosis with normal timing despite aphidicolin exposure (FIG. 3B). Thus, DNA synthesis by replicative DNA polymerase continues at least until 15-16 h post activation, concordant with the presence of spontaneous DNA damage foci. Incompletely replicated DNA delays entry into mitosis in an ATR- and CHK1-dependent manner.

To determine whether CHK1 kinase controls the timing of mitotic entry, we used both ICSI as well as parthenogenetic activation and incubated 1-cell embryos in one of two different CHK1 inhibitors, starting at 15 h. CHK1 inhibition advanced mitotic entry by 4 h, from 24 to 20.3 h in fertilized zygotes and from 21.7 to 17.25 h in parthenotes to as early as 16 h (FIG. 3C). CHK1 inhibition in G2 phase was detrimental to genome stability: 9/9 stained zygotes formed abnormal nuclei marked by 7H2AX foci after mitotic exit and failed cytokinesis. Although most control 2-cell embryos were euploid (12/16 blastomeres), CHK1 inhibition specifically in G2 phase resulted in a high degree of aneuploidy that was further amplified through the addition of aphidicolin (FIG. 3D). When CHK1 was inhibited in day-3 cleavage-stage embryos of 7-8 cells, no cavitation occurred (0/9 embryos), while 3/5 control embryos did undergo cavitation on day 6 of development (p<0.05, Fisher's exact test). CHK1 inhibition in cleavage-stage embryos also resulted in cells containing multiple micronuclei with γH2AX foci (results not shown).

### Example 4—Asymmetric Sister Fork Progression, Low Replication Fork Speed and G2 Replication in the First Cell Cycle

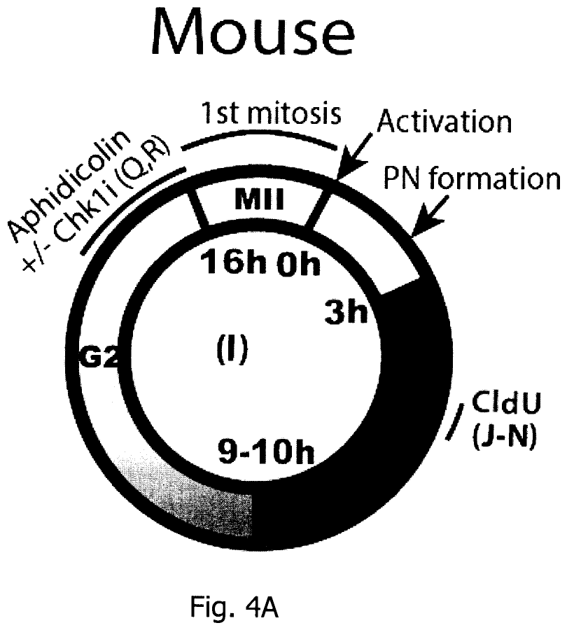

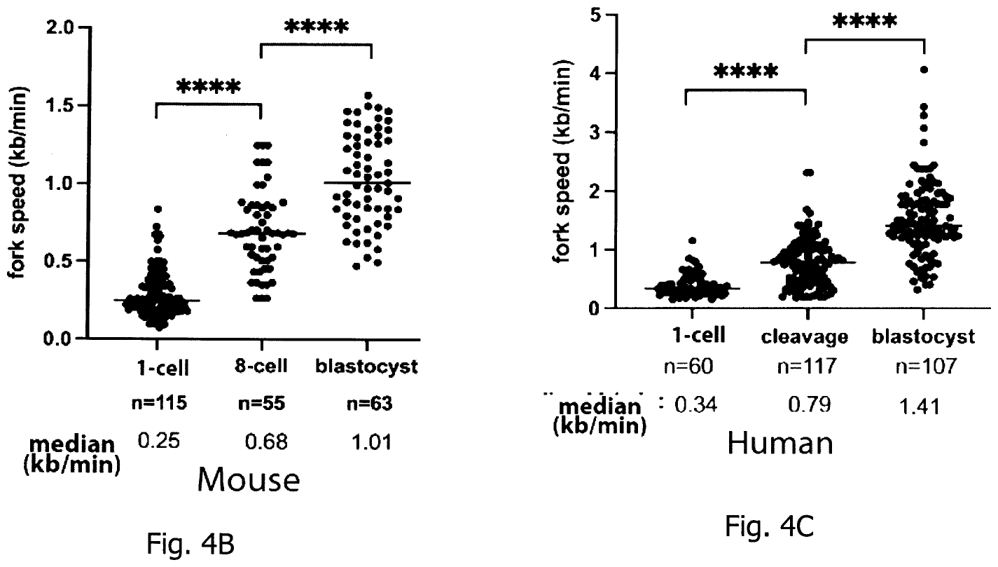

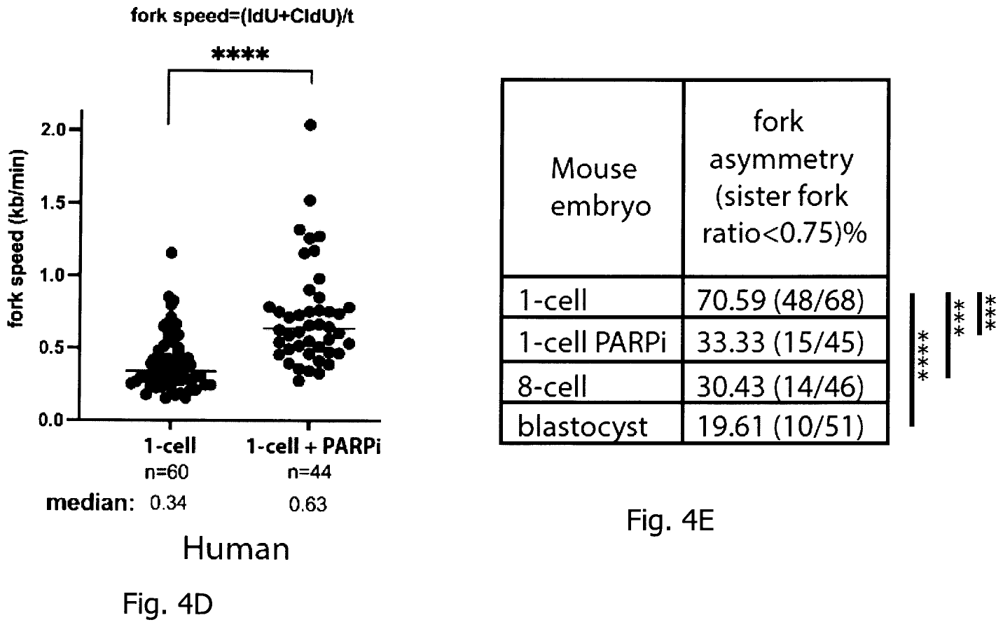

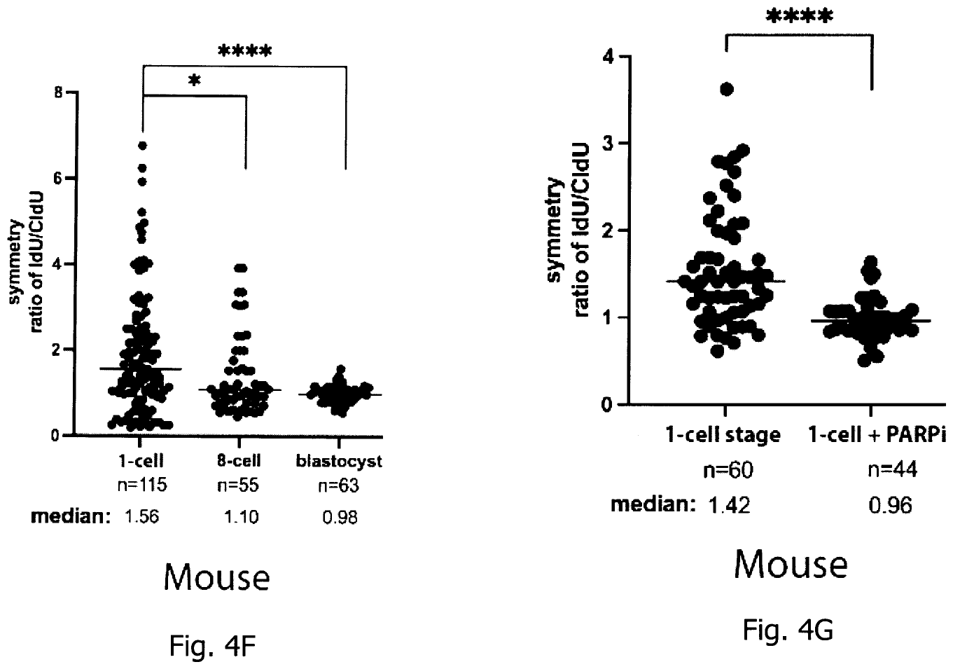

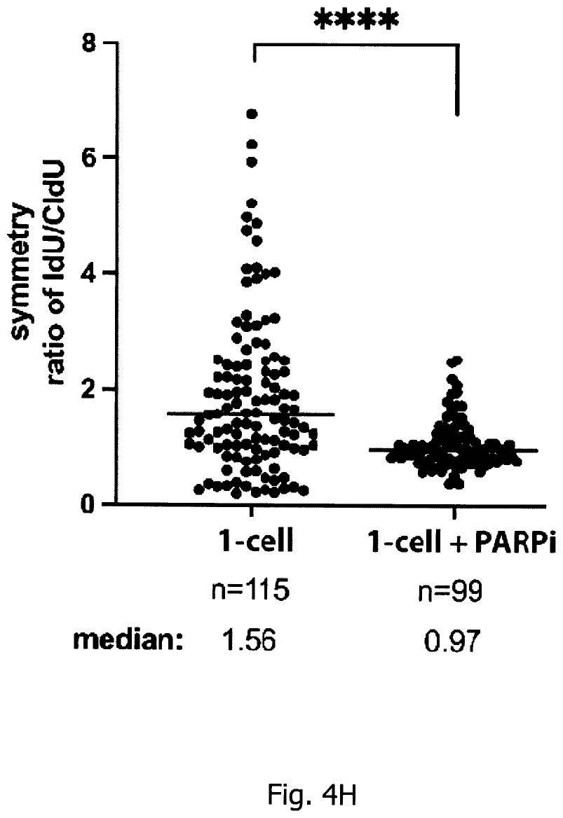

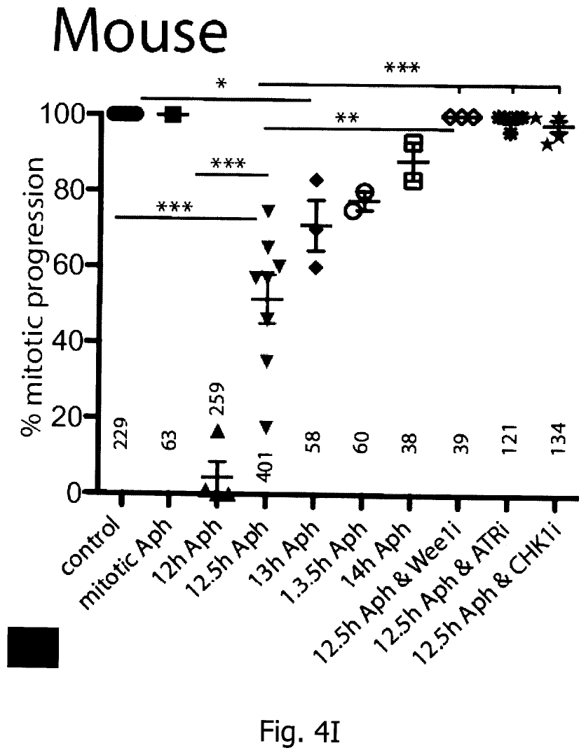

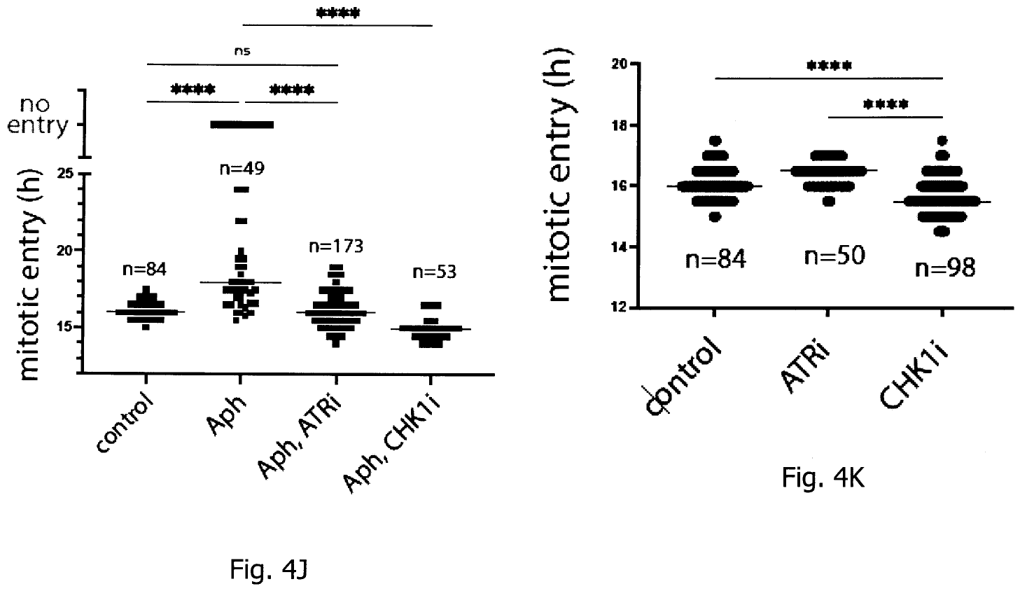

To better understand the first S phase in mammalian zygotes, we also used mice. Murine DNA replication occurs between 4 and 10 h post fertilization (Bui et al., 2010). Consistent with previous studies on fertilized zygotes, parthenogenetic embryos showed no detectable EdU staining between 10-12 h (n=10) and 12-14 h (n=10) post activation. Mitotic entry of controls occurred on average at 16 h, defining a G2 phase of approximately 6 h (FIG. 4A). As EdU staining of whole zygotes may not detect small segments of labeled DNA, we performed DNA fiber analysis of zygotes incubated in late G2 phase with IdU. IdU incorporation was seen in short segments that were estimated to be several hundred base pairs to approximately 1 kb in length (results not shown). These could be termination events, repair of collapsed forks, or gap-filling events.

To evaluate DNA replication fork progression in S phase, we performed sequential labeling using IdU and CldU pulses in S phase. Mouse and human embryos showed very slow replication fork progression at the 1-cell stage, which doubled at the cleavage stage and at the blastocyst stage (FIGS. 4B and 4C). Replication fork speed was increased through PARP inhibition in humans and mice (FIG. 4D). PARP inhibition increases replication fork progression and reduces fork reversal under replication stress (Zellweger et al., 2015). Frequent asymmetry was found in diverging sister forks, in particular at the zygote stage, indicating fork stalling (FIG. 4E). Asymmetry in track length was common in zygotes, less common in 8-cell embryos, and uncommon in blastocyst embryos in mice (FIG. 4F). PARP inhibition restored symmetry at the 1-cell stage in both mouse and human (FIGS. 4G and 4H). These data are consistent with DNA replication stress.

As in humans, aphidicolin application in G2 phase of mouse 1-cell embryos inhibited mitotic entry depending on the time point of application (FIG. 4I). G2 arrested zygotes formed foci with phosphorylated RPA32 on S33, S4, and S8 and foci for γH2AX and Rad51, which formed in an ATR-dependent manner within 2-4 h of fork stalling and resolving within 1-2 h after release from the compound. Both G2 arrest and delay of mitotic entry were dependent on G2 checkpoint kinases CHK1 and ATR (FIGS. 41 and 4J). CHK1 inhibition in G2 phase also advanced mitotic entry of mouse 1-cell embryos relative to untreated controls from 16.1 to 15.7 h post activation (FIG. 4K).

Notably, aphidicolin-treated human parthenotes reproduced defects recently described in human and bovine embryos associated with an abnormal first mitosis (Cavazza et al., 2021), including a failure to polarize chromosomes in the nucleus (16/17 nuclei in this study). Asynchrony in chromosome condensation and mitotic progression between two nuclei in two 2PN human zygotes was also observed (results not shown).

### Example 5—Spontaneous DSBs Result in Segmental Chromosome Loss in Human Cleavage-Stage Embryos

Unreplicated sites and DSBs can result in chromosome breakage upon entry into mitosis (reviewed in Mankouri et al. [2013]). Chromosomal analysis provides a means to map sites of spontaneous chromosome breakage, which could provide insight into the location of DNA damage and the causes of aneuploidy.

We first analyzed germinal vesicle (GV) oocytes using single cell genome sequencing. 11/11 GV oocytes showed normal chromosomal content without losses or gains of whole chromosomes or chromosomal arms. In contrast, embryos with Cas9-mediated DSBs showed segmental chromosomal changes at sites targeted by the gRNA, validating methods of analysis.

We analyzed eight 2-cell embryos and identified two segmental aneuploidies in 16 blastomeres. Thus, chromosome breakage is not the secondary consequence of a prior abnormal mitosis and can occur spontaneously at the first cell division consistent with the presence of unreplicated DNA and markers of DNA breakage in G2 zygotes.

We then isolated blastomeres from 26 thawed day-3 cleavage-stage embryos. 145 intact single cells were successfully thawed, collected, and sequenced of which 99 (68%) were aneuploid and 46 (32%) were euploid, and 5/26 embryos (28 cells, 19.5%) showed uniform chromosomal gains or losses, consistent with meiotic origin (FIG. 5A). 71 cells (49%) were aneuploid due to abnormalities acquired in mitosis characterized by different chromosome content in different cells of the same embryo, and 10 cells contained aneuploidies of both meiotic and mitotic origin. We identified a total of 301 chromosomal abnormalities due to mitotic segregation errors, consisting of 97 chromosomes with segmental aneuploidies (32%) with a copy-number change of a chromosome arm (1-2 copy-number transitions), 149 whole-chromosome gains and losses (50%), and 55 events (18%) with 3-10 copy-number transitions on the same chromosome, indicating fragmentation (FIG. 5B).

Mitotic aneuploidies in day-3 blastomeres showed a net loss of DNA-both segmental losses and whole-chromosome losses outnumbered gains (FIG. 5C). This contrasts with chromosomal errors in meiosis, where the ratio of gains/losses approximates 1 throughout preimplantation development (Franasiak et al., 2014) (FIG. 5C). Interestingly, for Cas9-induced chromosome segregation errors, the number of losses also exceeded gains in two different studies (FIG. 5C).

### Example 6—Spontaneous Chromosome Break Sites Map to Gene-Poor Regions and Centromeres

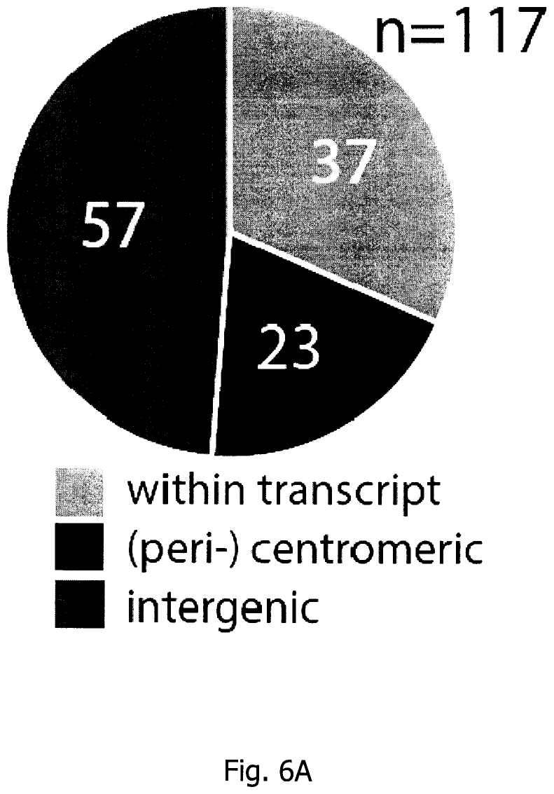

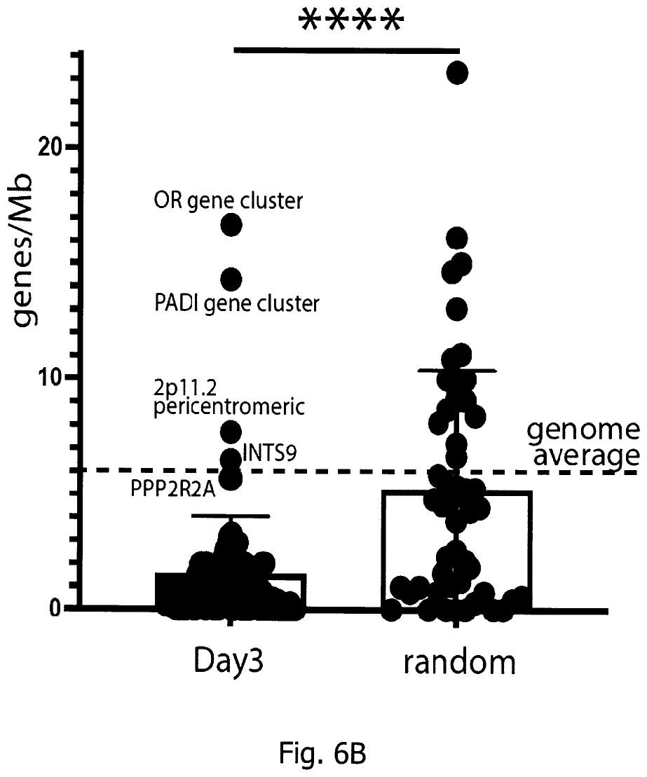

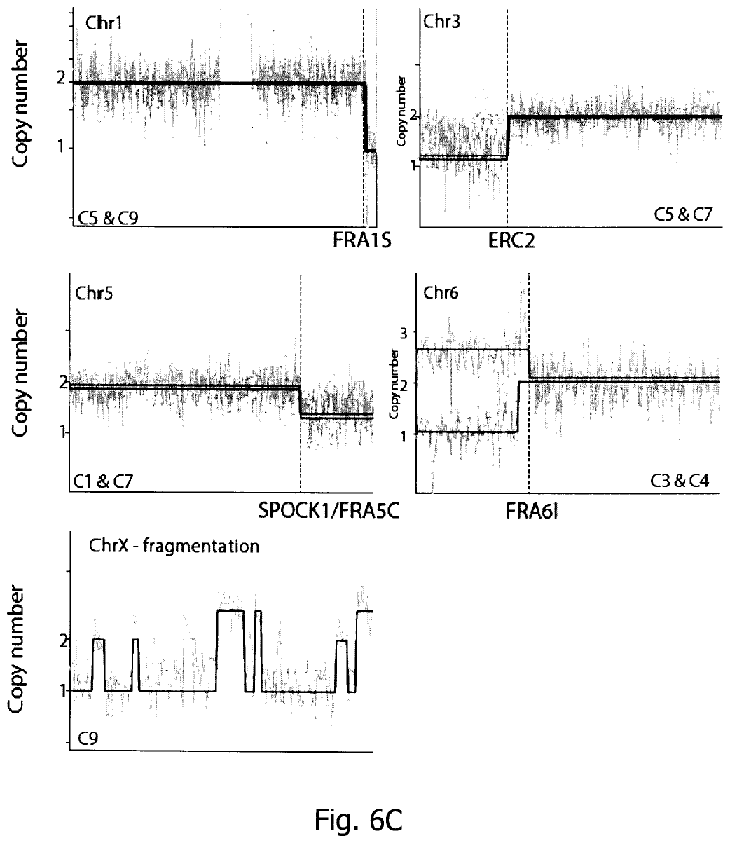

To determine the location of spontaneous chromosomal breaks, we identified regions of copy-number transition. A 2-fold drop or a 50% increase in read number across a chromosomal arm identified the location of a segmental break. We identified a total of 117 chromosomal breaks in blastomeres of day-3 embryos. Of these, 57 mapped to intergenic areas, 23 mapped to centromeric and pericentromeric regions, and 37 mapped to gene bodies (FIG. 6A). Concordant with the coordinates of chromosomal breaks in day-3 embryos, 7H2AX foci marking DNA breaks were found at centromeres, while the majority located to chromosomal arms. Gene density at break points was on average 1.5 genes/Mb, lower than at 50 randomly selected sites (FIG. 6B; p<0.0001). Only 5 sites had a gene density at genome average (6 genes/Mb) or higher, of which two were gene clusters.

Cleavage-stage embryo #4 is representative of the pattern of breakage: segmental breaks were observed in all (9/9) blastomeres, with reciprocal and mirroring copy-number transitions. Reciprocal events arise from asymmetric chromosome segregation at mitosis, leading to loss and gain in daughter cells (e.g., FRA6I in FIG. 6C). Mirroring changes arise from cell-cycle progression of a cell, with aneuploidy acquired in a prior mitosis (e.g., at SPOCK1 in FIG. 6C). The division of human blastomeres with broken chromosomes is also seen after Cas9-induced breaks. Each of the mirroring or reciprocal breaks located to gene-poor regions and common fragile sites (FIG. 6C). Segmental losses occurred on chromosome 1 at FRA1S containing the RYR2 gene, at chromosome 3p14.3 containing the ERC2 gene, at FRA5C on chromosome 5 within the SPOCK1 gene, at FRA6I on chromosome 6 near the centromere, and at FRA11O on chromosome 11 within the CNTN5 gene. On the X chromosome, a break at the centromere appears to have preceded the fragmentation of the whole chromosome. Interestingly, transcripts encoded by genes located at these break points were all very long, with SPOCK1 spanning 524 kb, RYR2 792 kb, and ERC2 960 kb and CNTN5 spanning 1,289 kb. No whole-chromosome aneuploidies were observed in this embryo, and thus, chromosome breakage was not the secondary consequence of aneuploidy.

### Example 7—Micronuclei Constitute Mitotic Aneuploidies

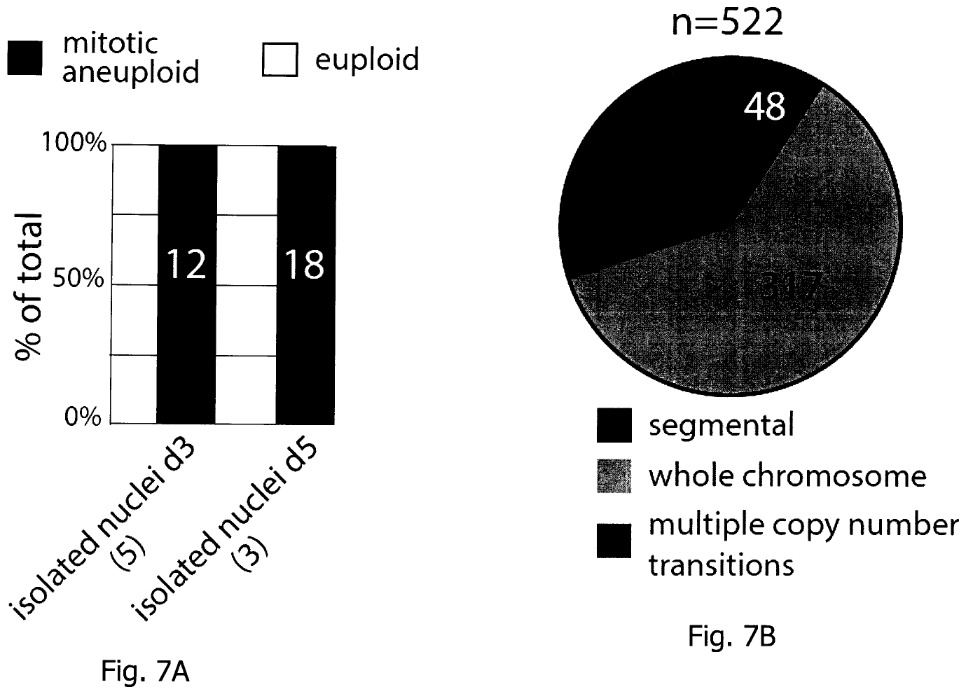

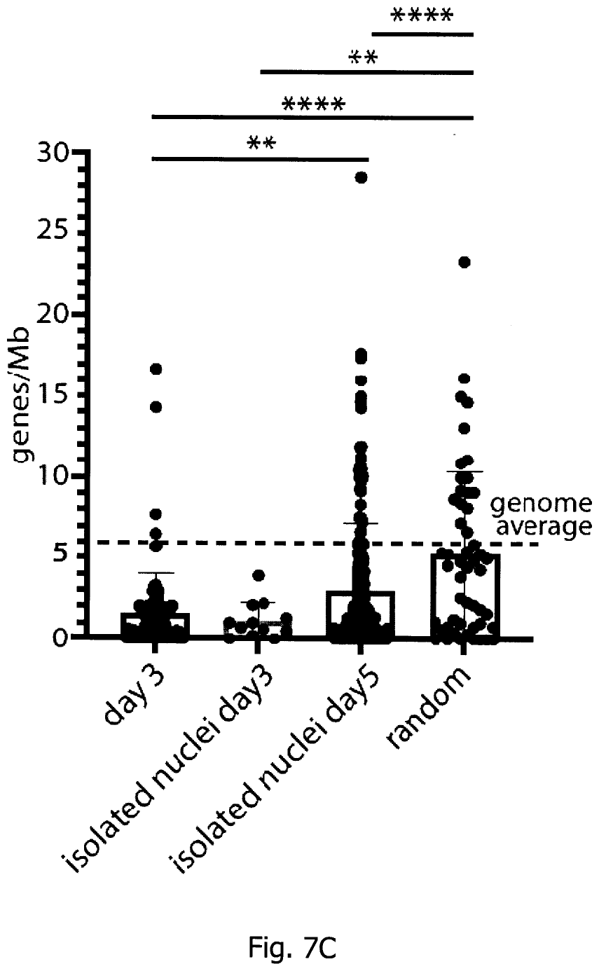

The sequencing-based analysis of intact cells provides incomplete resolution, as it is an aggregate signal of different nuclei within the same cell. To circumvent this limitation, we determined the chromosomal content of individually isolated micronuclei. We sequenced 12 individual micronuclei from day-3 blastomeres as well as 18 micronuclei from spontaneously arrested day 5 multicell embryos. All micronuclei were aneuploid (n=30) (FIG. 7A), including both segmental, whole-chromosome errors, and fragmented chromosomes (FIG. 7B). Isolated micronuclei typically were nullisomic for most chromosomes and contained as little as a single chromosome segment. Break-point mapping in micronuclei isolated from day-3 embryos showed that all (n=16) mapped to gene-poor areas (average 1.1 genes/Mb), with gene density lower than random (FIG. 7C). 12 break points mapped to chromosomal arms, while 4 (25%) mapped to centromeric and pericentromeric regions. In micronuclei isolated from arrested multicell day 5 embryos, 251 break points were mapped. Although gene density at break-point sites was below genome average and random simulated breakage (FIG. 7C), there was an increase in gene-rich sites (FIG. 7C). This increase may be due to the instability of chromosomes in micronuclei (Crasta et al., 2012), resulting in secondary break points.

### Example 8—Chromosomal Fragility is Independent of Embryonic Genome Activation

Including all break sites identified in day-3 blastomeres, in micronuclei from day-3, and arrested day 5 embryos, we mapped a combined 466 break sites of which 371 were independent events. 154 (42%) were recurrent, locating to 55 different genomic locations. Recurrence was defined as a breakage in the same genomic region in different embryos. For instance, independent breaks were observed in blastomeres (2 independent breaks) and in micronuclei (2 independent breaks) at the CNTN5 locus and its neighboring intergenic region. 22 fragile regions were defined by three or more independent recurrences of which 9 (consisting of 40 independent events) mapped to centromeres and pericentromeric regions, whereas 13 (consisting of 48 independent events) mapped to chromosomal arms.

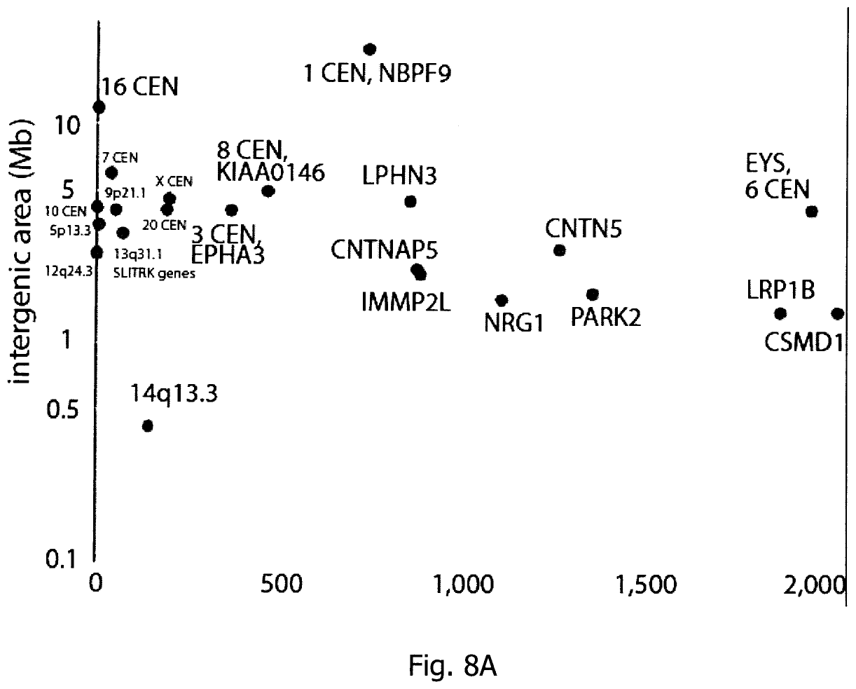

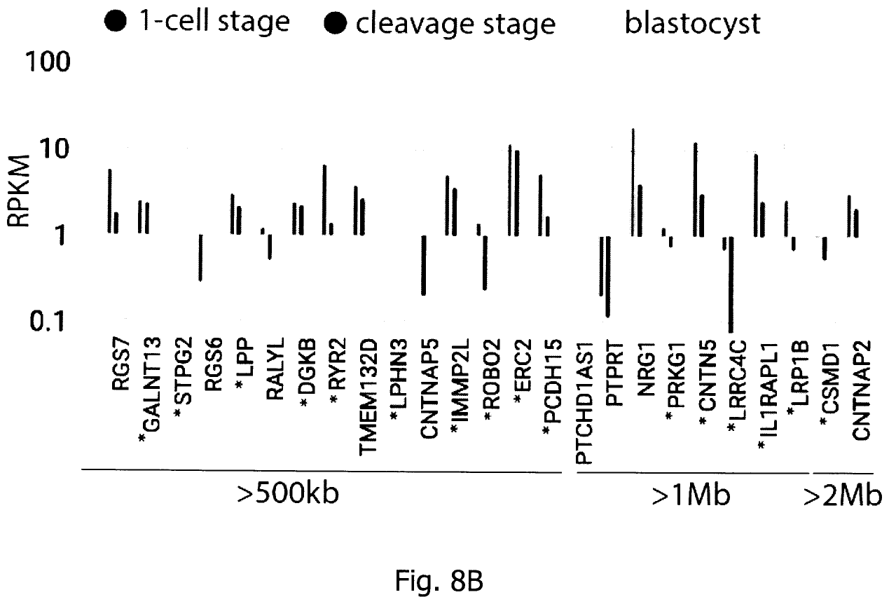

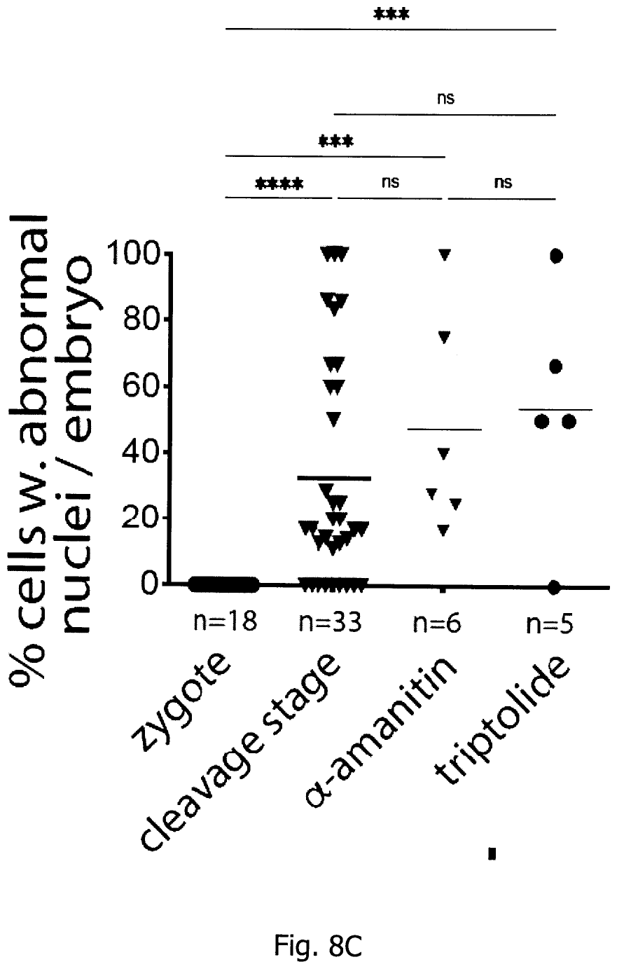

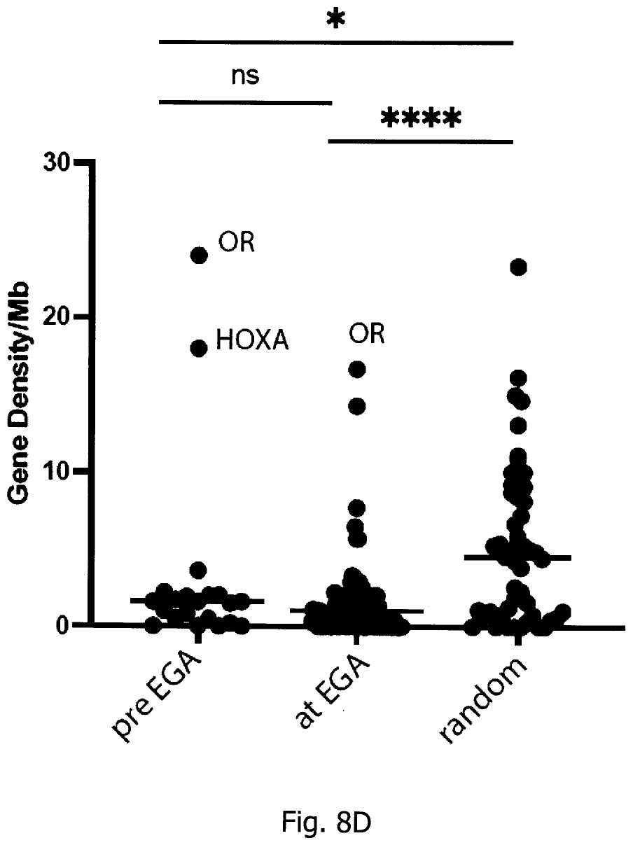

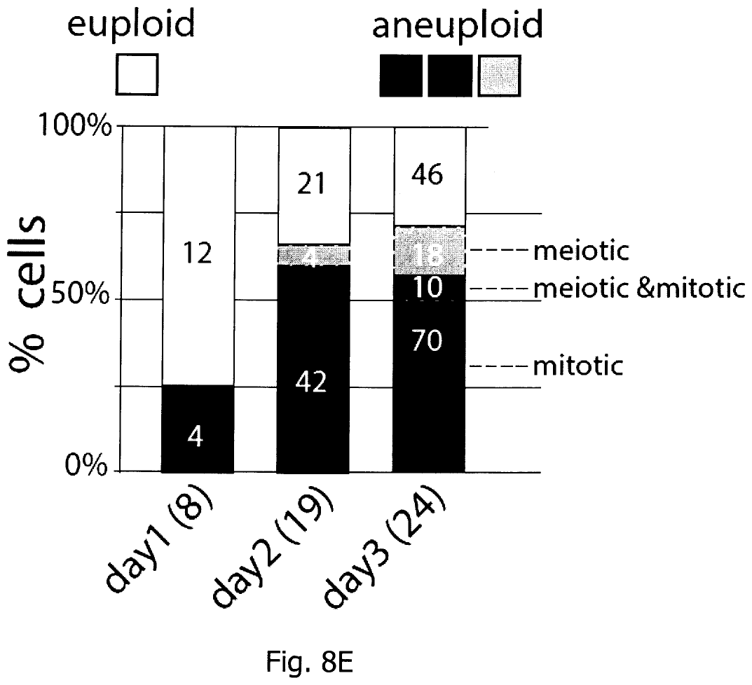

Fragile regions with recurrent breaks harbored either a long transcript or overlapped a large intergenic area (FIG. 8A). 18 of 22 mapped to aphidicolin-sensitive fragile sites identified in somatic cells (Mrasek et al., 2010). Chromosome fragility in somatic cells is associated with the expression of long genes in late replicating regions of the genome (Wei et al., 2016), potentially because of conflicts between transcription and replication (Helmrich et al., 2011; Brison et al., 2019; Hamperl et al., 2017).

We determined the expression of long transcripts at the location of recurrent chromosome breaks using published gene expression datasets (Yan et al., 2013). Long fragile site transcripts showed low levels in oocytes and were nearly undetectable at the cleavage stage (FIG. 8B). Likewise, only 3/37 transcripts of any length that harbored a break site in day-3 cleavage-stage embryos showed upregulation from the 1-cell stage to the cleavage stage. Among those 3 transcripts are RAPGEF2 (92-kb transcript), SP3 (57-kb transcript), and NBPF10, which has annotated transcript isoforms of 10 or 1,174 kb. Thus, the expression of neither long nor short transcripts correlated with chromosome fragility in the cleavage-stage embryo.

To directly determine if transcription contributes to embryonic DNA damage and abnormal chromosome segregation in preimplantation embryos, we incubated 2PN zygotes from days 1 to 3 in the presence of a-amanitin (n=6), an inhibitor of RNA polymerase II, or triptolide, an inhibitor of both RNA polymerase I and II (n=5). Cell-cycle progression of human embryos until the 4-8-cell stage does not require transcription, which is inhibited by a-amanitin (Braude et al., 1988; Egli et al., 2011). None of the embryos incubated with triptolide (0/4) showed ethynyl uridine label incorporation, whereas controls did, confirming transcriptional inhibition (results not shown). Moreover, no mouse embryos (0/15) developed beyond the two-cell stage in the presence of triptolide, indicating the effective inhibition of EGA, which occurs at the 2-cell stage in mice. Independent of the transcriptional inhibitor, micronucleation, which reflects mitotically acquired aneuploidies, remained comparable with untreated cleavage-stage embryos (FIG. 8C). Furthermore, blastomeres contained foci for both γH2AX, RAD51, and RPA32. The presence of RPA32 foci in mitosis directly demonstrates cell-cycle progression with incompletely replicated single-stranded DNA (results not shown).

To determine the location of chromosome breakage prior to EGA, we analyzed the chromosome content of 8 embryos on day 1 of development at the 2-cell stage and 19 embryos on day 2 of development, 17 of which were at the 4-cell stage. 2 segmental errors were found in 2-cell embryos within a day of fertilization, and 8 independent segmental errors were found in 67 blastomeres of day-2 embryos. 8 of these were in gene-poor regions, whereas 2 were gene-rich, including the HOXA gene cluster on chromosome 7 and the olfactory receptor gene cluster pericentromeric on chromosome 11. Furthermore, 11 mirroring chromosomal changes in the same day-3 embryo arise through sequential divisions of an error that arose prior to EGA. Gene density at a total of 21 break sites occurring prior to EGA was lower than at simulated random sites (FIG. 8E), and the pattern of fragility prior to EGA was the same as at EGA in day-3 embryos. Aneuploidy due to abnormal mitosis was observed in products of all three cleavage divisions on days 1-3 of development, including before EGA (FIG. 8E).

### Example 9—Sites of G2 Replication are Gene Poor and Concordant with Spontaneous Chromosome Breakage Site

In somatic cells, gene-poor regions are prone to fragility due to late replication in the cell cycle and entry into mitosis with unreplicated DNA (Le Beau et al., 1998). Notably, 16/25 long fragile site transcripts greater than 500 kb show mitotic DNA synthesis in somatic cell lines (Macheret et al., 2020; Ji et al., 2020) (starred genes in FIG. 8B), suggesting that the timing of DNA replication completion may contribute to chromosome fragility of the human embryo.

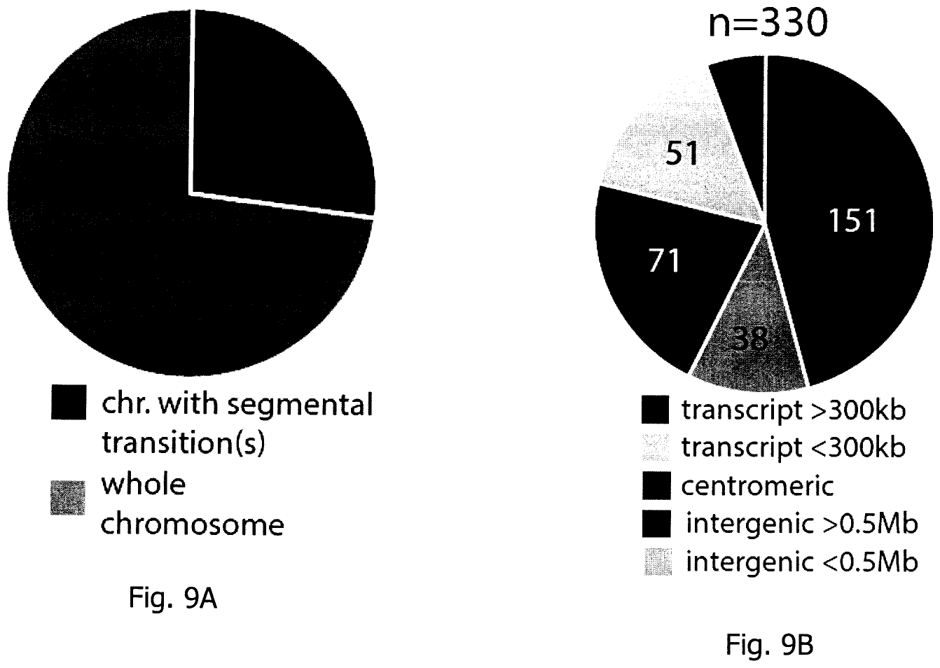

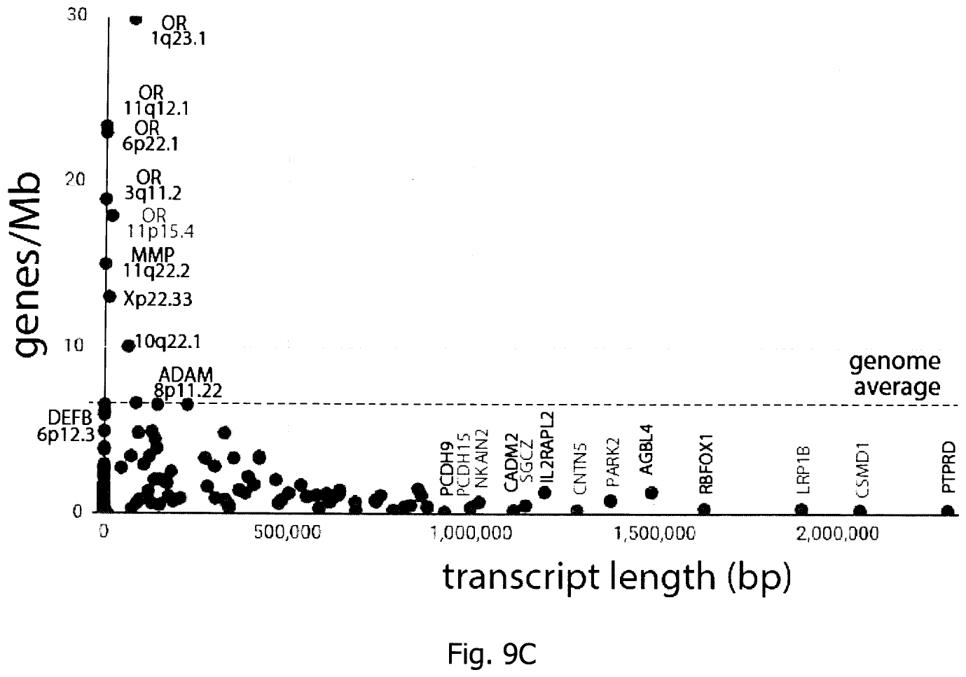

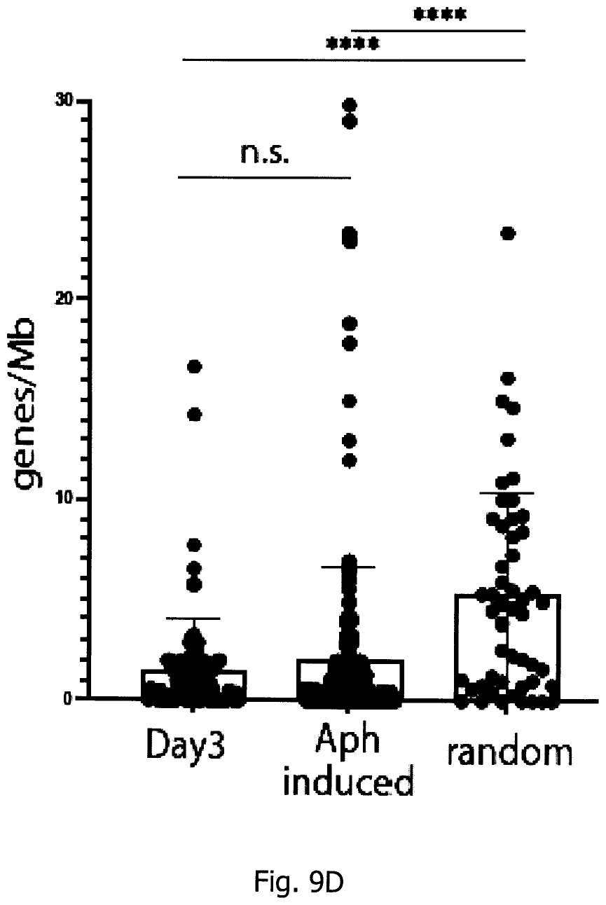

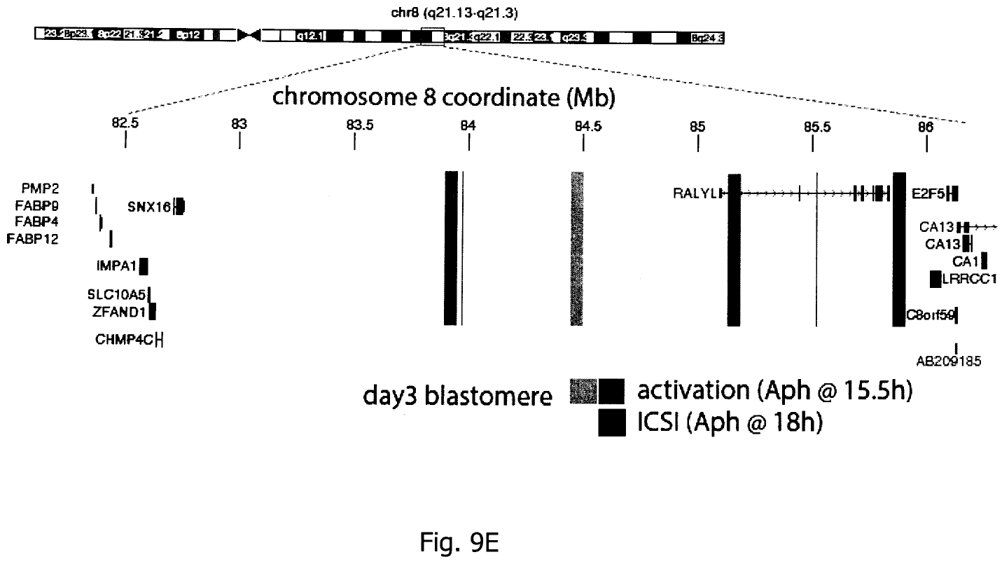

To map sites of late replication completion in human zygotes, we incubated 6 parthenogenetically activated human embryos with aphidicolin and G2 checkpoint kinase inhibitor at 15.5 h into the first cell cycle, uniformly resulting in the formation of multinucleate cells. Individual micronuclei (n=25) were isolated, and 21 from 4 zygotes were successfully sequenced. All contained incomplete genomes. The most common aneuploidy was whole-chromosome losses (248 chromosomes, 73%), followed by chromosomes with one or more segmental copy-number transitions (93 chromosomes, 27%) (FIG. 9A).

The break-point analysis identified a total of 331 break sites. The analysis of polar bodies of the same embryos was performed to exclude a single break of meiotic origin. Of the 330 mitotic break sites, 191 (58%) showed reciprocity in a different nucleus, whereas 139 were singular, consistent with a recovery of chromosomal material per embryo of 59%. Per embryo, an average of 51 independent break points were found, corresponding to at least 51 unreplicated sites. 19 break sites (6%) were centromeric, and 189 break sites were intergenic of which 151 (46%) were located in intergenic areas of 0.5 Mb or greater. 122 break sites (37%) located within a transcript of which 71 (21.5%) were longer than 300 kb (FIG. 9B). Among the longest genes were PARK2, CNTN5, PTPRD, CSMD1, MDGA2, PCDH9, and PCDH15 (FIG. 9C). 184 breaks (56%) were in gene-poor areas of 1 or fewer genes/Mb, and 24 sites (7%) had a gene density at and above the genome average (approximately 6.9 genes/Mb). Of these 24 gene-rich sites, 16 (5%) located to gene clusters, in particular to olfactory receptor gene clusters (11), metalloproteinase gene clusters MMP (1) and ADAM (1), as well as CYP4 (1) and DEFB (2) gene clusters (FIG. 9C). The remaining gene-rich areas had adjacent gene-poor regions of more than 0.5 Mb (e.g., SLC30A7 at 1p21.1). Only one site on chromosome 9q31.3 was not within or immediately adjacent to a gene-poor region. When comparing induced break sites with random simulated breakage, gene density was lower (p=1.5e-7) (FIG. 9D). Gene density at aphidicolin-induced break sites and spontaneous breakage in day-3 cleavage-stage embryos were not different (FIG. 9D).

Break sites induced by aphidicolin at sites of G2 replication were concordant with spontaneous break sites, such as at CNTN5 on chromosome 11 or RALYL on chromosome 8 (FIGS. 8A and 9E). Of the 22 fragile regions in untreated embryos, 14 (64%) were also found in the cohort of aphidicolin-induced breaks. To evaluate concordance with fertilized embryos, we also incubated 4 2PN zygotes with aphidicolin and CHK1 inhibitor at 18 h post fertilization by ICSI. 7 chromosomal breaks were identified in 4 different genomic locations of which 2 were concordant with break points in parthenogenetically activated oocytes at the centromere on chromosome 9 and at RALYL on chromosome 8 (FIG. 9E).

Taken together, DNA synthesis continues at least until 18 h of the first cell cycle and into mitotic prophase. Notably, aphidicolin exposure at 18 h did not delay mitotic progression (FIG. 3B), despite the presence of at least 2 unreplicated sites per embryo.

To evaluate the potential role of transcription in aphidicol insensitive replication delays, we examined the expression of transcripts at break sites (Yan et al., 2013). All but four of 93 transcription units with break sites, EIF3E, UVRAG, GULP1, and CRADD, were either not expressed or transcript levels decreased from the 1-cell stage to the morula stage after EGA. Only 3 of 330 break sites (0.9%) located near a transcription start site, and only one of these, CRADD, was expressed at a later developmental stage. Furthermore, we compared embryo break sites with transcription-dependent replication delays described in somatic cells (Sarni et al., 2020). Of the 119 sites identified by Sarni and colleagues, 6 sites were shared at the genes FTO, LAMA2, MAGI2, SCAPER, THSD4, and VPS13B. However, zygotes are transcriptionally largely silent, and none of these genes showed appreciable transcription after EGA. We also compared embryo break sites to aphidicolin-induced recurrent break sites in actively transcribed genes of human neuronal progenitors (Wang et al., 2020). Of 31 sites identified by Wang and colleagues, 10 were shared with the embryo (32%) at CTNNA2, NRG3, RBFOX1, LRP1B, PARK2, MAGI2, LINGO2, DLG2, PCHD15, and LRRD4C. Eight of these ten genes show mitotic DNA synthesis in somatic cells (Ji et al., 2020; Macheret et al., 2020). Again, these genes showed no expression during cleavage development. Thus, the need for DNA synthesis in G2 phase in human zygotes is independent of transcription-replication conflicts.

### Example 10—Fragility in Late Replicating Regions Compromises Developmental Potential

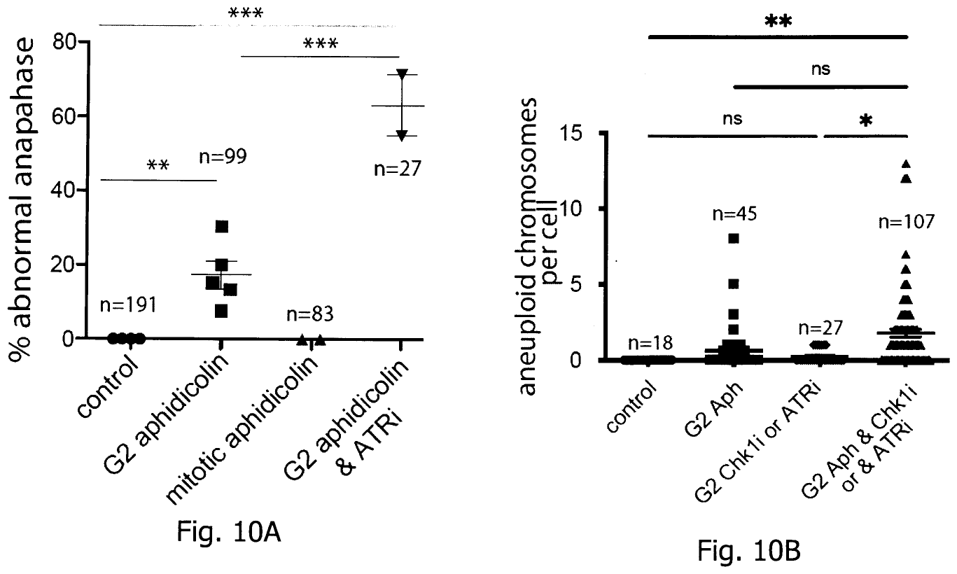

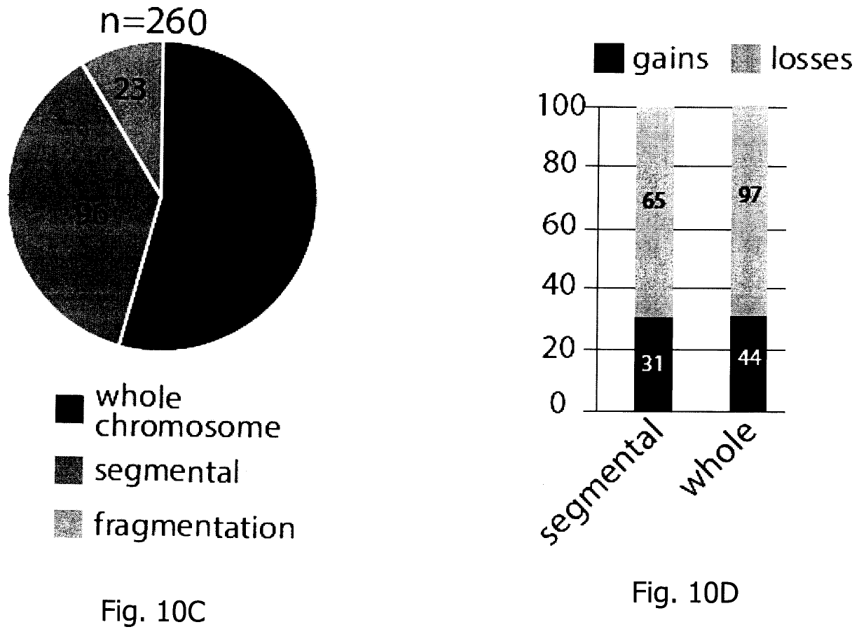

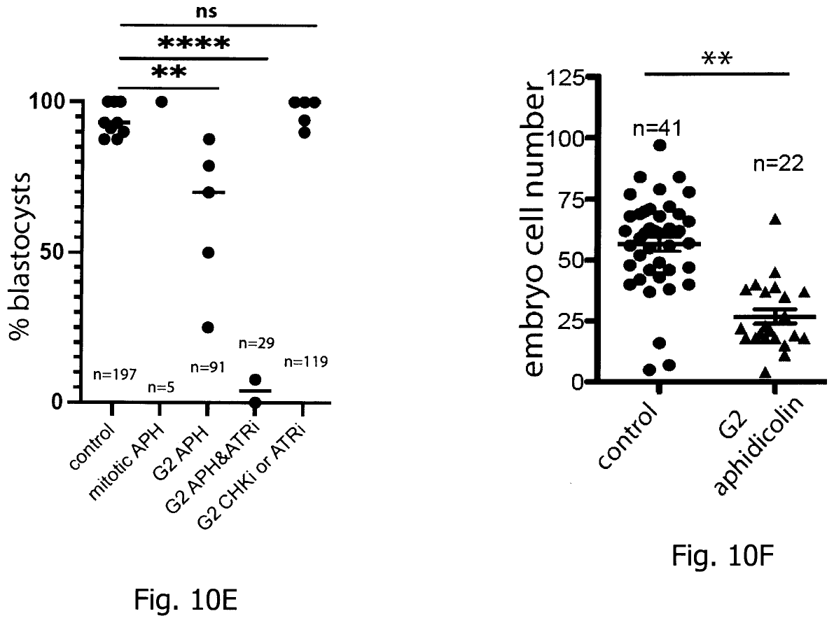

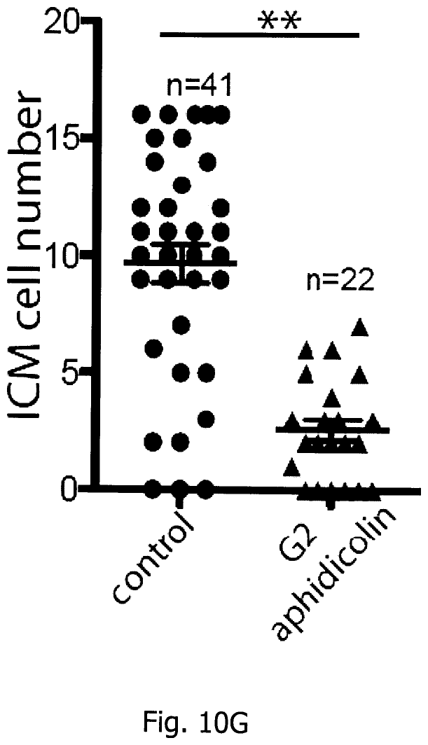

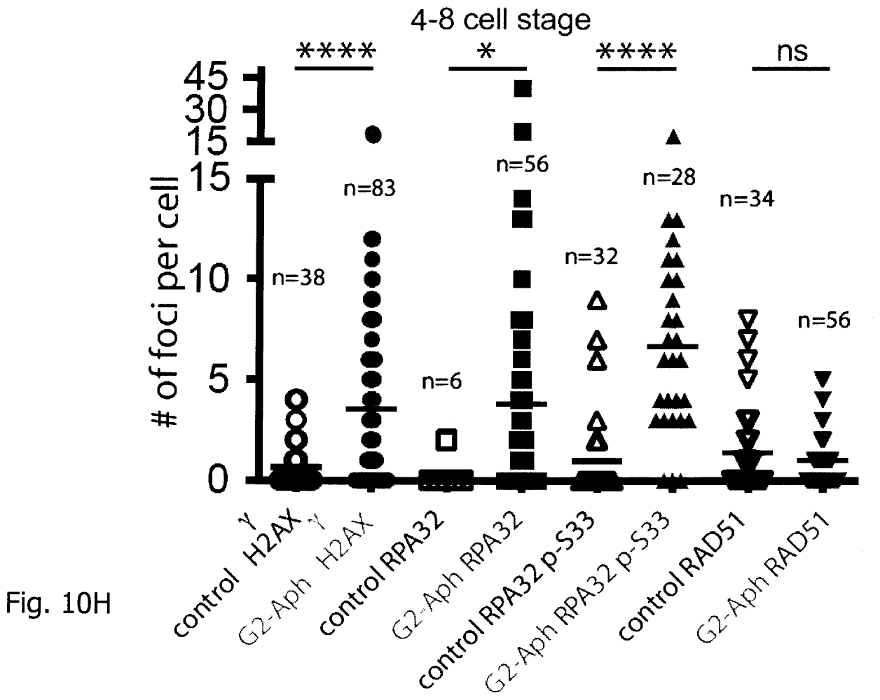

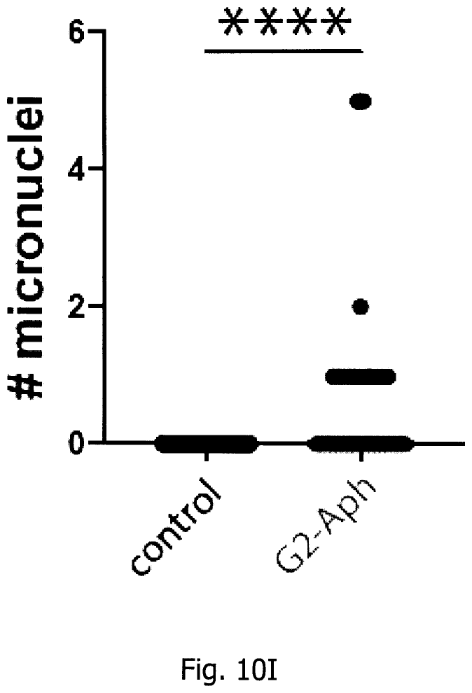

In mice, spontaneous mitotic chromosome segregation errors are less common than in human embryos (Bolton et al., 2016), and preimplantation development is more efficient. Because of the low frequency of spontaneous segregation errors, the mouse lends itself to experimental interference. To determine whether inhibiting G2 replication reproduces the chromosomal and developmental abnormalities seen in human embryos, we applied the DNA polymerase inhibitor aphidicolin in G2 phase. All 191/191 control parthenogenetic zygotes showed normal chromosome segregation at the first mitosis, whereas 17/99 (17.4%, range per experiment 7%-30%) treated embryos showed abnormal anaphase with lagging chromosomes and chromosome fragments (FIG. 10A). Aphidicolin applied only after pronuclear envelope breakdown was inconsequential (FIG. 10A). The frequency of abnormal anaphase figures increased to 63% (16/27) when embryos were exposed to both ATR kinase inhibitor and aphidicolin in G2 phase.

Chromosome content analysis of sister blastomeres at the 2-cell stage showed that 98% of control cells had normal mitotic segregation (57/58), whereas 33% (14/45) of aphidicolin-treated blastomeres showed aneuploidy. The combination of aphidicolin and G2 checkpoint inhibition further increased aneuploidy to 64.5% of the blastomeres (69/107), consistent with the frequency of abnormal anaphases. In aphidicolin-treated samples, a total of 28 chromosomal abnormalities were found in 45 blastomeres, or 0.62 per cell. G2 checkpoint inhibition alone resulted in 0.3 abnormalities per cell (8 aneuploidies in 27 cells). In embryos treated with both aphidicolin and G2 checkpoint inhibitor, aneuploidies increased to 2.1 per cell (226 aneuploidies in 107 cells) (FIG. 10B). Karyotype abnormalities included whole-chromosome segregation errors (54%), segmental errors (37%), and chromosome fragmentation (9%) (FIG. 10C). Whole chromosome as well as segmental losses exceeded gains by a ratio of approximately 1:2 (FIG. 10D).

Although in controls, 92% of eggs developed to the blastocyst stage, of the cleaved embryos exposed to aphidicolin in G2 phase, 72% developed to the blastocyst stage (range 25%-87%) (FIG. 10E), concordant with the frequency of euploid embryos. On day 5 of development, blastocysts obtained after treatment with aphidicolin in G2 showed reduced total cell number and fewer ICM cells marked by Pou5f1, indicating low embryo quality (FIGS. 10F and 10G). Aphidicolin-treated embryos showed increased markers of DNA damage, including γH2AX, Rpa32, and Rpa32 phosphoS33 at the 4-8 cell stage at 48 h post activation (FIG. 10H). Embryos were developmentally delayed and showed excluded blastomeres, binucleation, and increased micronucleation (FIG. 10I). Thus, incomplete replication at the first mitosis destabilizes the genome in subsequent cell divisions and phenocopies common spontaneous defects in human embryos.

### Example 11—Response to Unreplicated DNA Differs Between Mouse and Human Embryos

In comparison to human cleavage-stage embryos, untreated mouse embryos showed not only a lower frequency of spontaneous chromosome segregation errors (FIG. 10B versus FIG. 8E) but also a lower frequency of segregation errors caused by interference with replication completion in G2 phase (FIG. 9A). Surprisingly, 38 of 107 (35.5%) of mouse 2-cell embryos were euploid despite combined aphidicolin exposure and G2 checkpoint inhibition, whereas all human embryos were aneuploid. Mouse embryos showed aneuploidies on just 2 chromosomes per cell, whereas human embryos showed catastrophic aneuploidy with on average 51 break sites or more than one genomic alteration per chromosome. This difference could be due to a lower number of unreplicated sites in mice at the time of inhibitor application or due to differences in the response and resolution of unreplicated DNA.

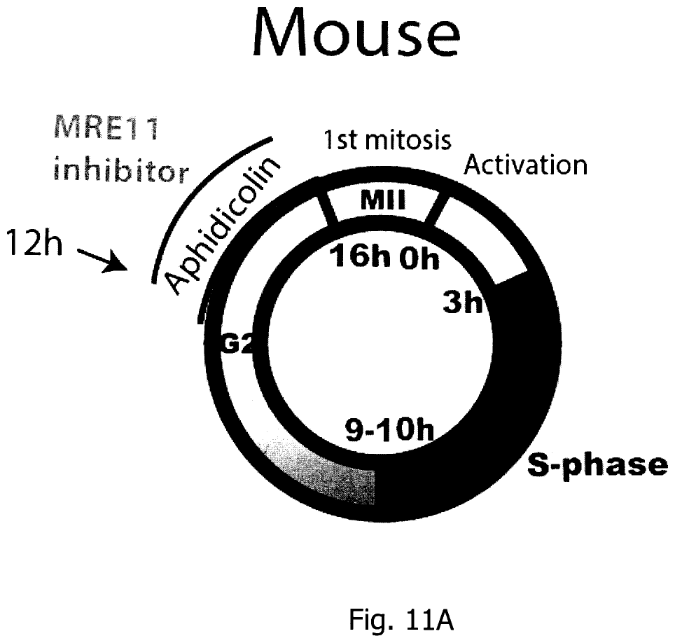

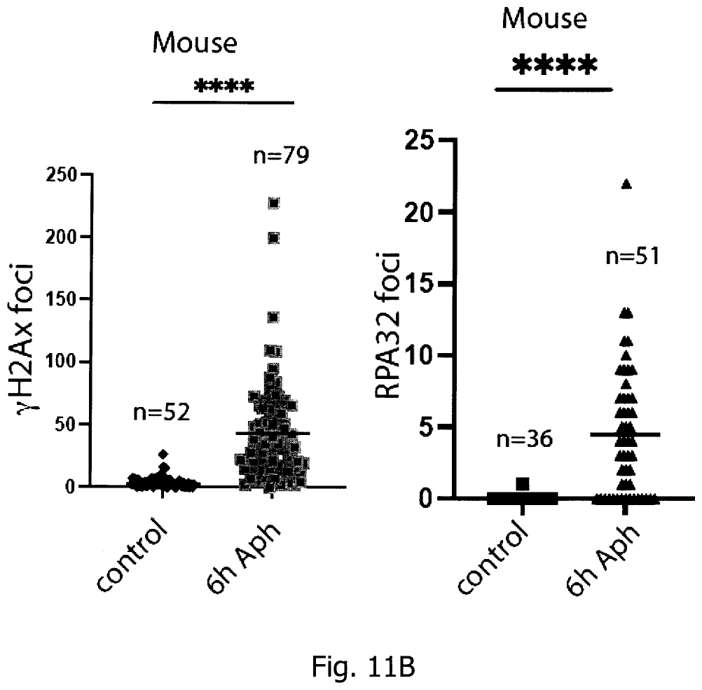

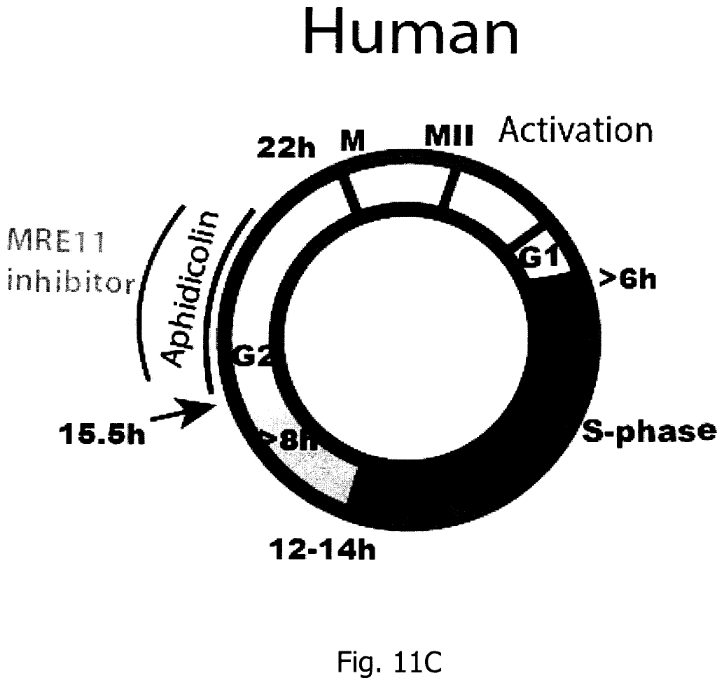

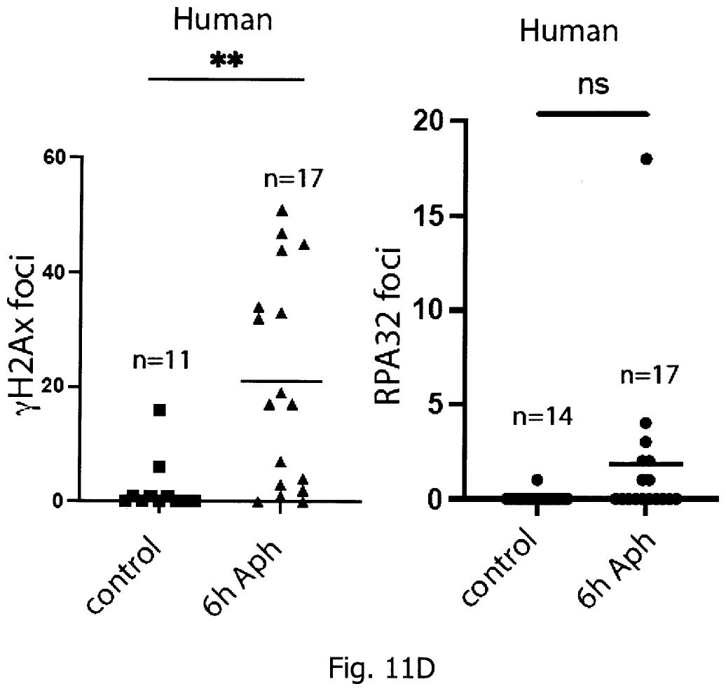

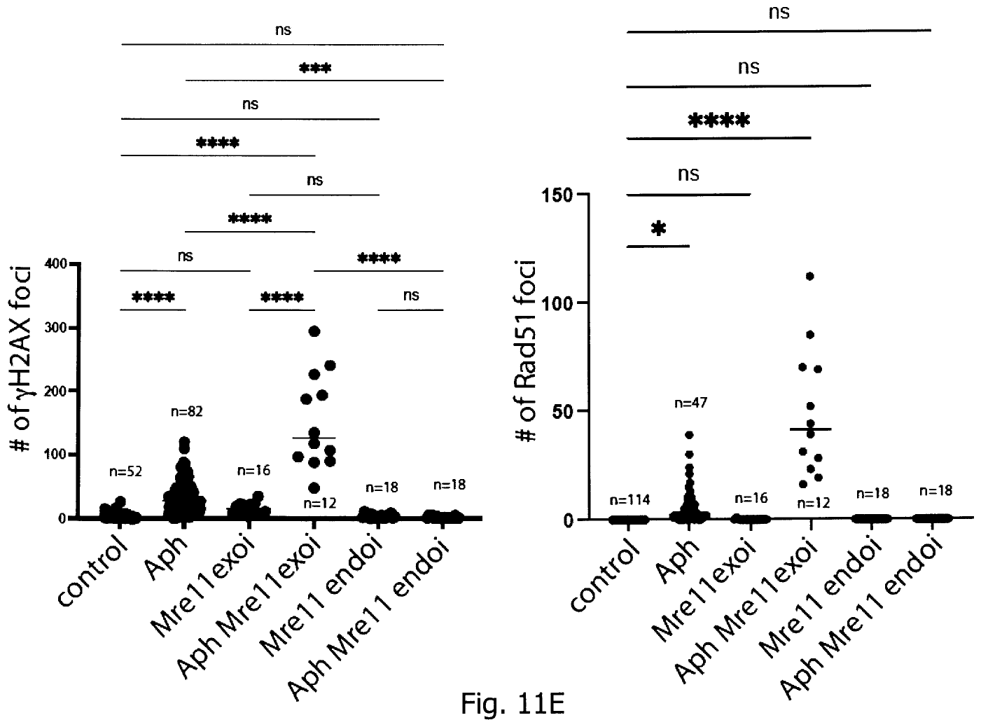

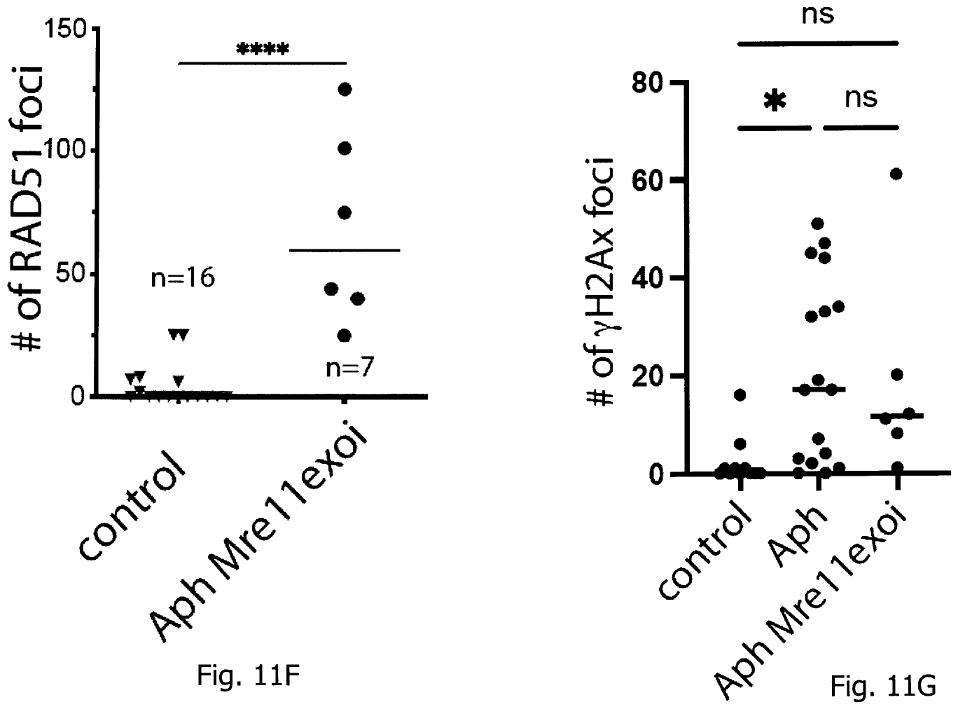

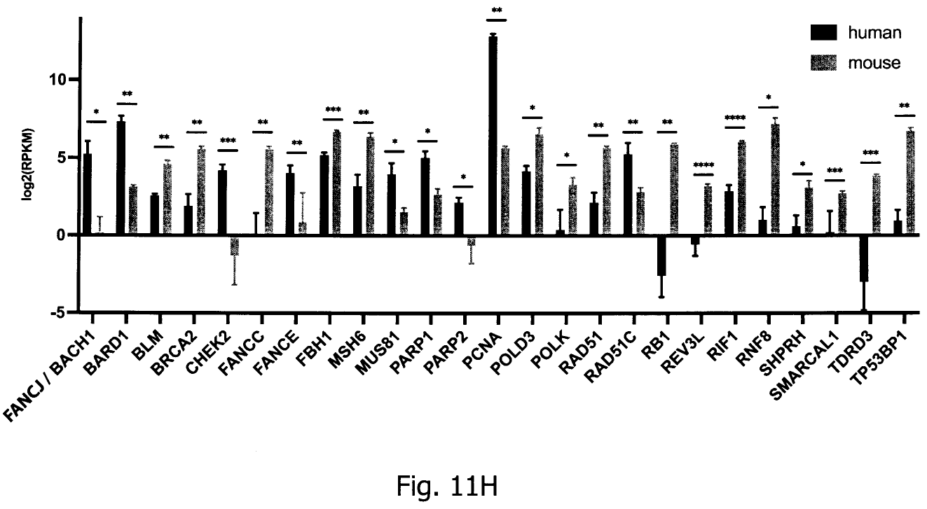

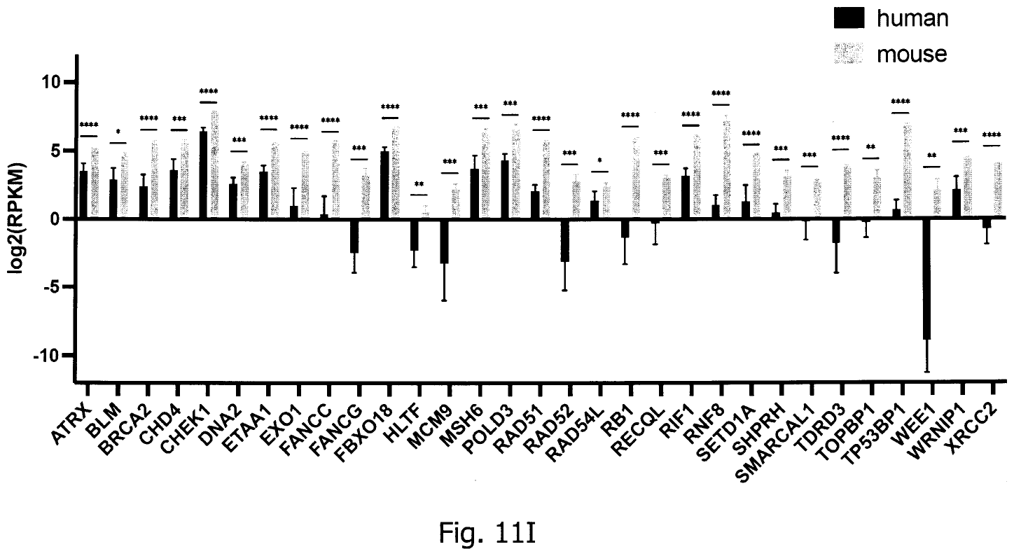

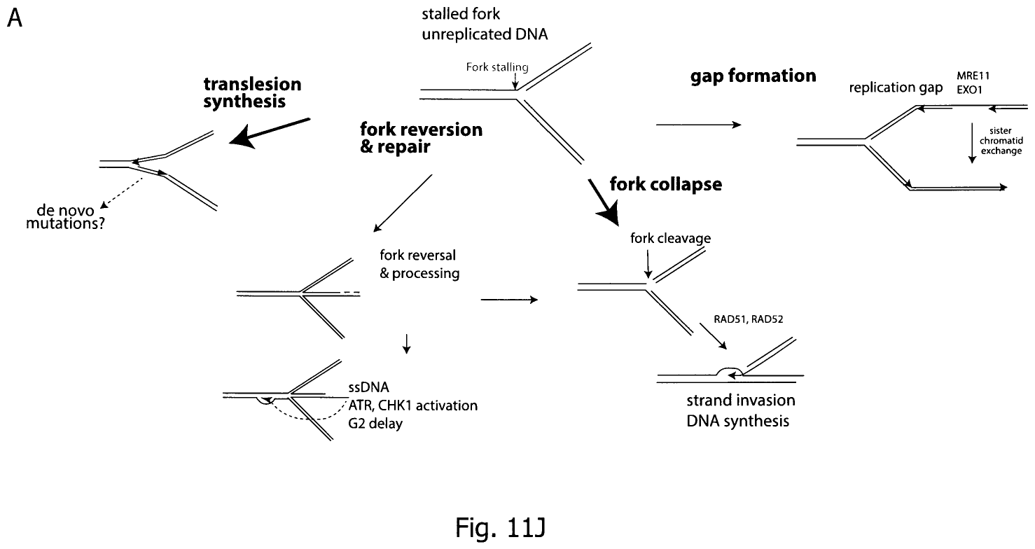

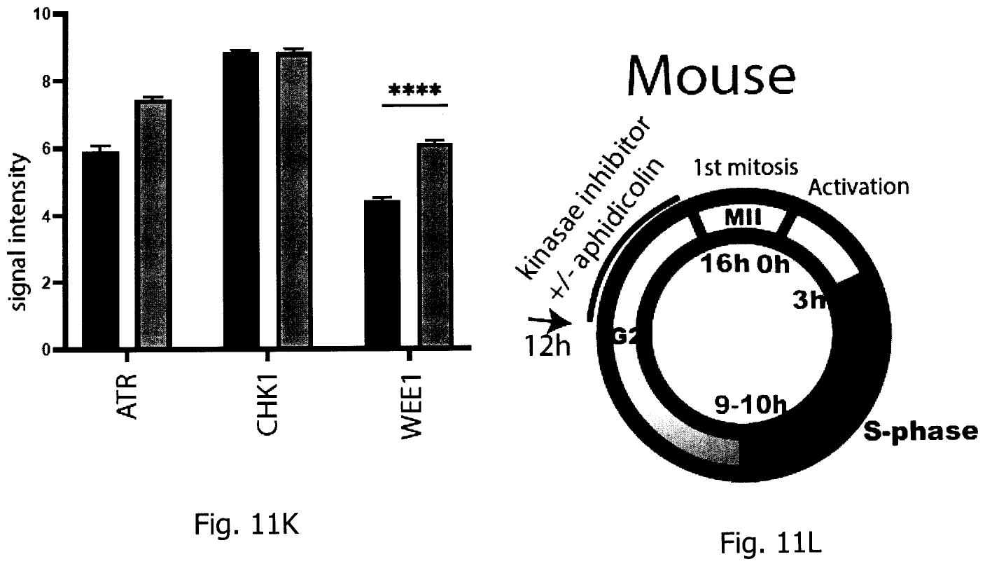

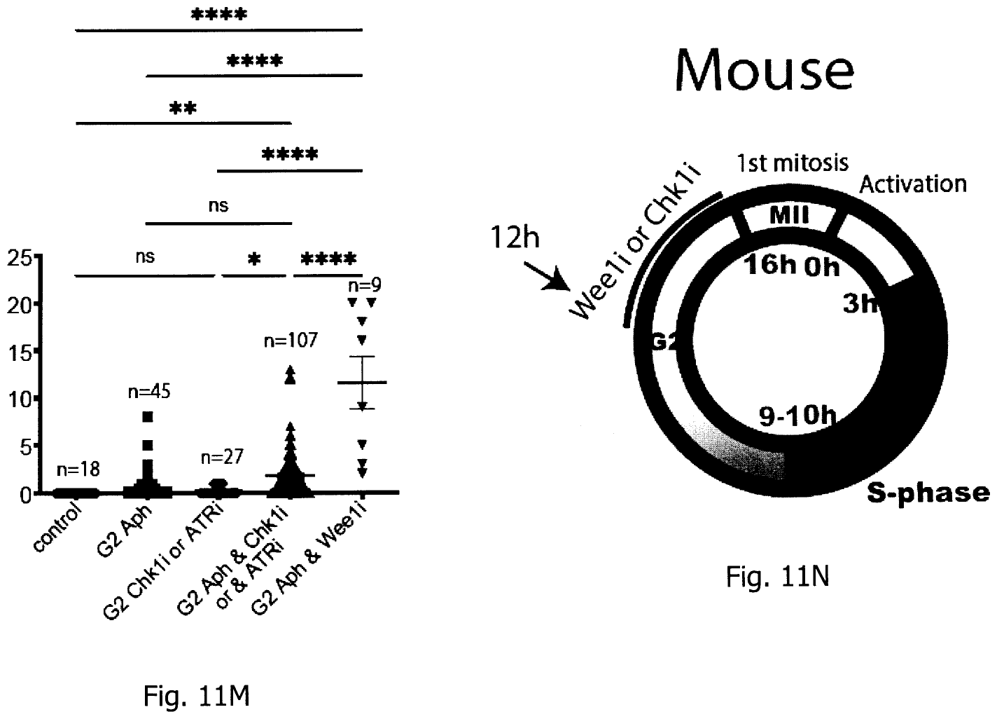

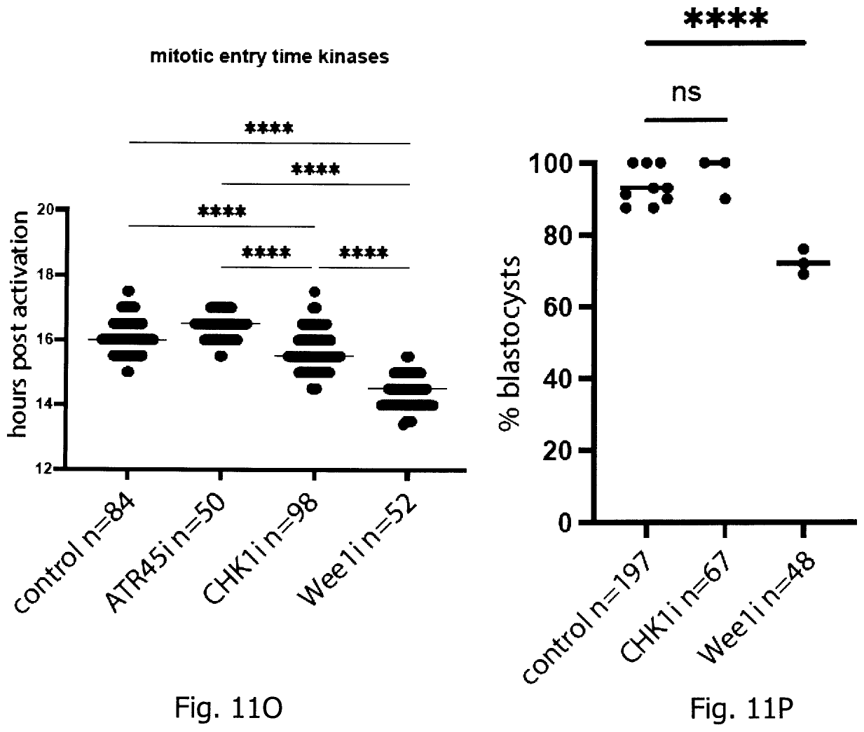

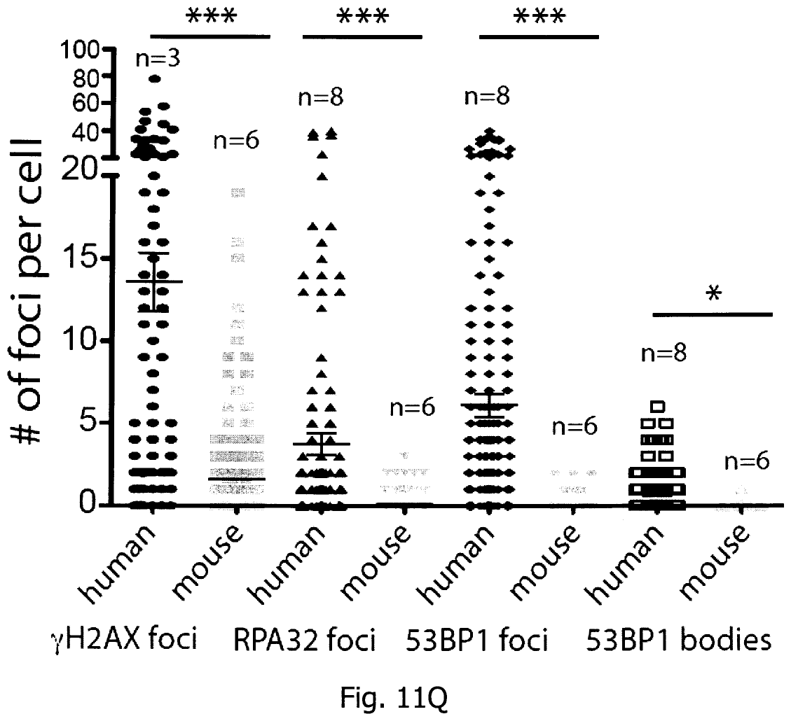

To estimate the number of unreplicated sites, we counted the number of DNA damage foci induced in G2 phase (FIGS. 11A-11G). Aphidicolin incubation resulted in a higher number of γH2AX and RPA32 foci in mice (mean γH2AX foci 43, range 0-228, in mice versus human, mean 21, range 0-51). Approximately 10% of γH2AX foci were positive for RPA32 in both species, which averaged 1.8 in human versus 4.5 in mice per haploid nucleus (FIGS. 11B and 11D). The processing of DSBs and the extension of ssDNA gaps required for the formation of RPA binding and ATR kinase activation involves the nuclease activities of MRE11. The inhibition of MRE11 exonuclease using mirin results in a repair defect at DSBs and thereby increases the number of DNA damage foci (Dupre' et al., 2008; Shibata et al., 2014). The addition of mirin combined with aphidicolin (schematic in FIG. 11A) increased the number of foci marked by γH2AX in mouse zygotes to more than 100 and the number of Rad51 foci to more than 50 (FIG. 11E). The inhibition of MRE11 endonuclease activity abrogated foci for both markers (FIG. 11E), suggesting that sites stalled by aphidicolin were processed in an MRE11-dependent manner. An increase in the number of RAD51 foci upon combined exposure to aphidicolin and mirin was also seen in human zygotes, increasing to 50 sites on average (FIG. 11F). The number of γH2AX foci was not increased by MRE11 inhibition beyond aphidicolin incubation alone, though it could be an underestimate as large H2Ax bodies may contain multiple sites (FIG. 11G). Thus, the number of foci marking unreplicated sites in G2 phase is higher in mice than in human at the relevant time points. The number of aneuploidies induced by aphidicolin incubation exceeds the number of γH2AX foci in human, whereas in mice it is the reverse. Thus, human zygotes are less efficient at forming DNA damage foci and more efficient at converting sites of incomplete replication to chromosomal breaks and aneuploidy.

Consistent with differences in the processing of stalled replication forks, transcripts of SMARCAL1, FBH1, RAD51, and Bloom (BLM) were lower in human than in mice, whereas FANCJ/BACH1 was expressed at higher levels (FIGS. 11H and 11I). These factors are involved at different steps of replication fork reversion and resection (reviewed in Joseph et al. [2020]) (FIG. 11J). In contrast, transcripts for MUS81, which cleaves replication intermediates at prophase of mitosis to form DSBs, were higher in human (FIGS. 11H and 11I). Interestingly, human zygotes progressed to mitotic prophase (16 of 17) with condensed chromatids despite persistent γH2AX and RPA32 foci, whereas mouse zygotes arrested in G2 phase without chromosome condensation (results not shown). Human cleavage embryos were reported to express low levels of WEE1 kinase (Kiessling et al., 2010), which acts upstream of CDK1 and MUS81 to inhibit cleavage of replication forks that remain stalled late in the cell cycle (Duda et al., 2016). We confirmed low WEE1 transcript levels at the 1-cell stage in human relative to mice (FIG. 11K). When Wee1 kinase inhibitor was added alongside aphidicolin to mouse embryos, mitosis resulted in a highly fragmented genome (FIGS. 11L and 11M), reproducing the frequent segmental breakages seen in human embryos. Wee1 kinase inhibition in G2 phase mouse embryos advanced mitosis by 1.5 h and impaired developmental potential compared with untreated controls (FIGS. 11N-11P).

In summary, inefficient DNA damage foci formation, progression to mitotic prophase with unreplicated DNA, and frequent formation of chromosomal breaks at sites of incomplete replication, followed by an unstable genome during cleavage divisions (FIG. 11Q) are defining differences between human and mouse preimplantation development.

### REFERENCES

Ahuja, et al. 2016. A short G1 phase imposes constitutive replication stress and fork remodelling in mouse embryonic stem cells. Nat Commun, 7, 10660.
Anantha, et al. 2007. Sequential and synergistic modification of human RPA stimulates chromosomal DNA repair. J Biol Chem, 282, 35910-23.
Baslan, et al. 2015. Optimizing sparse sequencing of single cells for highly multiplex copy number profiling. Genome Res, 25, 714-24.
Bester, et al. 2011. Nucleotide deficiency promotes genomic instability in early stages of cancer development. Cell, 145, 435-46.
Bohgaki, et al. 2011. Genomic instability, defective spermatogenesis, immunodeficiency, and cancer in a mouse model of the RIDDLE syndrome. PLoS Genet, 7, e1001381.
Bolton, et al. 2016. Mouse model of chromosome mosaicism reveals lineage-specific depletion of aneuploid cells and normal developmental potential. Nat Commun, 7, 11165.
Braude, et al. 1988. Human gene expression first occurs between the four- and eight-cell stages of preimplantation development. Nature, 332, 459-61.
Brison, et al. 2019. Transcription-mediated organization of the replication initiation program across large genes sets common fragile sites genome-wide. Nat Commun, 10, 5693.
Bui, et al. 2010. Effect of trichostatin A on chromatin remodeling, histone modifications, DNA replication, and transcriptional activity in cloned mouse embryos. Biol Reprod, 83, 454-63.
Casante, et al. 2022. Fifteen years of autologous oocyte thaw outcomes from a large university-based fertility center. Fertility and Sterility, 118, 158-66.
Cavazza, et al. 2021. Parental genome unification is highly error-prone in mammalian embryos. Cell, 184, 2860-2877.e22.
Charrier, et al. 2011. Discovery of potent and selective inhibitors of ataxia telangiectasia mutated and Rad3 related (ATR) protein kinase as potential anticancer agents. J Med Chem, 54, 2320-30.
Crasta, et al. 2012. DNA breaks and chromosome pulverization from errors in mitosis. Nature, 482, 53-8.
Danecek, et al. 2021. Twelve years of SAMtools and BCFtools. Gigascience, 10.
Doil, et al. 2009. RNF168 binds and amplifies ubiquitin conjugates on damaged chromosomes to allow accumulation of repair proteins. Cell, 136, 435-46.
Duda, et al. 2016. A Mechanism for Controlled Breakage of Under-replicated Chromosomes during Mitosis. Dev Cell, 39, 740-755.
Dupre, et al. 2008. A forward chemical genetic screen reveals an inhibitor of the Mre11-Rad50-Nbs1 complex. Nat Chem Biol, 4, 119-25.
Egli, et al. 2011. Reprogramming within hours following nuclear transfer into mouse but not human zygotes. Nat Commun, 2, 488.
Ercilla, et al. 2020. Physiological Tolerance to ssDNA Enables Strand Uncoupling during DNA Replication. Cell Rep, 30, 2416-2429.e7.
Fox, C., et al., Local and systemic factors and implantation: what is the evidence? Fertil Steril, 2016. 105(4): p. 873-84
Franasiak, et al. 2014. The nature of aneuploidy with increasing age of the female partner: a review of 15,169 consecutive trophectoderm biopsies evaluated with comprehensive chromosomal screening. Fertil Steril, 101, 656-663.el.
Hamperl, et al. 2017. Transcription-Replication Conflict Orientation Modulates R-Loop Levels and Activates Distinct DNA Damage Responses. Cell, 170, 774-786.e19.
Harrigan, et al. 2011. Replication stress induces 53BP1-containing OPT domains in G1 cells. J Cell Biol, 193, 97-108.
Hassold, T. and P. Hunt, To err (meiotically) is human: the genesis of human aneuploidy. Nat Rev Genet, 2001. 2(4): p. 280-91.
Helmrich, et al. 2011. Collisions between replication and transcription complexes cause common fragile site instability at the longest human genes. Mol Cell, 44, 966-77.
Hirai, et al. 2009. Small-molecule inhibition of Wee1 kinase by MK-1775 selectively sensitizes p53-deficient tumor cells to DNA-damaging agents. Mol Cancer Ther, 8, 2992-3000.
Ikegami, et al. 1978. Aphidicolin prevents mitotic cell division by interfering with the activity of DNA polymerase-alpha. Nature, 275, 458-60.
Jackson and Pombo 1998. Replicon clusters are stable units of chromosome structure: evidence that nuclear organization contributes to the efficient activation and propagation of S phase in human cells. J Cell Biol, 140, 1285-95.
Ji, et al. 2020. Genome-wide high-resolution mapping of mitotic DNA synthesis sites and common fragile sites by direct sequencing. Cell Res, 30, 1009-1023.
Joseph, et al. 2020. Time for remodeling: SNF2-family DNA translocases in replication fork metabolism and human disease. DNA Repair (Amst), 95, 102943.
Kiessling, et al. 2010. Genome-wide microarray evidence that 8-cell human blastomeres over-express cell cycle drivers and under-express checkpoints. J Assist Reprod Genet, 27, 265-76.
King, et al. 2014. Characterization and preclinical development of LY2603618: a selective and potent Chk1 inhibitor. Invest New Drugs, 32, 213-26.
Kuo and Yang 2008. Gamma-H2AX—a novel biomarker for DNA double-strand breaks. In Vivo, 22, 305-9.
Larsson, et al. 2018. Computational correction of index switching in multiplexed sequencing libraries. Nat Methods, 15, 305-307.
Le Beau, et al. 1998. Replication of a common fragile site, FRA3B, occurs late in S phase and is delayed further upon induction: implications for the mechanism of fragile site induction.
Lemmen, J. G., et al., The total pregnancy potential per oocyte aspiration after assisted reproduction-in how many cycles are biologically competent oocytes available? J Assist Reprod Genet, 2016. 33(7): p. 849-54.
Li and Durbin 2009. Fast and accurate short read alignment with Burrows-Wheeler transform. Bioinformatics, 25, 1754-60.
Liaw, et al. 2011. DNA-PK-dependent RPA2 hyperphosphorylation facilitates DNA repair and suppresses sister chromatid exchange. PLoS One, 6, e21424.
Liu, et al. 2000. Chk1 is an essential kinase that is regulated by Atr and required for the G(2)/M DNA damage checkpoint. Genes Dev, 14, 1448-59.
Liu, et al. 2012. Distinct roles for DNA-PK, ATM and ATR in RPA phosphorylation and checkpoint activation in response to replication stress. Nucleic Acids Res, 40, 10780-94.
Lukas, et al. 2011. 53BP1 nuclear bodies form around DNA lesions generated by mitotic transmission of chromosomes under replication stress. Nat Cell Biol, 13, 243-53.
Macheret, et al. 2020. High-resolution mapping of mitotic DNA synthesis regions and common fragile sites in the human genome through direct sequencing. Cell Res, 30, 997-1008.
Mankouri, et al. 2013. How unfinished business from S-phase affects mitosis and beyond. Embo j, 32, 2661-71.
Mrasek, et al. 2010. Global screening and extended nomenclature for 230 aphidicolin-inducible fragile sites, including 61 yet unreported ones. Int J Oncol, 36, 929-40.
Munne, S., et al., Chromosome mosaicism in cleavage-stage human embryos: evidence of a maternal age effect. Reprod Biomed Online, 2002. 4(3): p. 223-32
Olshen, et al. 2004. Circular binary segmentation for the analysis of array-based DNA copy number data. Biostatistics, 5, 557-72.
Palmerola, et al. 2018. Low estradiol responses in oocyte donors undergoing gonadotropin stimulation do not influence clinical outcomes. J Assist Reprod Genet.
Pertea, et al. 2018. Thousands of large-scale RNA sequencing experiments yield a comprehensive new human gene list and reveal extensive transcriptional noise. bioRxiv, 332825.
Petermann, et al. 2010. Hydroxyurea-stalled replication forks become progressively inactivated and require two different RAD51-mediated pathways for restart and repair. Mol Cell, 37, 492-502.
Piberger, et al. 2020. PrimPol-dependent single-stranded gap formation mediates homologous recombination at bulky DNA adducts. Nat Commun, 11, 5863.
Raderschall, et al. 1999. Nuclear foci of mammalian recombination proteins are located at single-stranded DNA regions formed after DNA damage. Proc Natl Acad Sci USA, 96, 1921-6.
Sarni, et al. 2020. 3D genome organization contributes to genome instability at fragile sites. Nat Commun, 11, 3613.
Scheinin, et al. 2014. DNA copy number analysis of fresh and formalin-fixed specimens by shallow whole-genome sequencing with identification and exclusion of problematic regions in the genome assembly. Genome Res, 24, 2022-32.
Schultz, et al. 2000. p53 binding protein 1 (53BP1) is an early participant in the cellular response to DNA double-strand breaks. J Cell Biol, 151, 1381-90.
Shibata, et al. 2014. DNA double-strand break repair pathway choice is directed by distinct MRE11 nuclease activities. Mol Cell, 53, 7-18.
Shibata, et al. 2014. DNA double-strand break repair pathway choice is directed by distinct MRE11 nuclease activities. Mol Cell, 53, 7-18.
Tachibana, et al. 2013. Human embryonic stem cells derived by somatic cell nuclear transfer. Cell, 153, 1228-38.
Vassin, et al. 2009. Human RPA phosphorylation by ATR stimulates DNA synthesis and prevents ssDNA accumulation during DNA-replication stress. J Cell Sci, 122, 4070-80.
Wang, et al. 2020. Increased Neural Progenitor Proliferation in a hiPSC Model of Autism Induces Replication Stress-Associated Genome Instability. Cell Stem Cell, 26, 221-233.e6.
Ward and Chen 2001. Histone H2AX is phosphorylated in an ATR-dependent manner in response to replicational stress. J Biol Chem, 276, 47759-62.
Wei, et al. 2016. Long Neural Genes Harbor Recurrent DNA Break Clusters in Neural Stem/Progenitor Cells. Cell, 164, 644-55.
Wright, et al. 1994. Inhibitor analysis of calf thymus DNA polymerases alpha, delta and epsilon. FEBS Lett, 341, 128-30.
Yamada and Egli 2017. Genome Transfer Prevents Fragmentation and Restores Developmental Potential of Developmentally Compromised Postovulatory Aged Mouse Oocytes. Stem Cell Reports, 8, 576-588.
Yan, et al. 2013. Single-cell RNA-Seq profiling of human preimplantation embryos and embryonic stem cells. Nat Struct Mol Biol, 20, 1131-9.
Zabludoff, et al. AZD7762, a novel checkpoint kinase inhibitor, drives checkpoint abrogation and potentiates DNA-targeted therapies. Mol Cancer Ther, 7, 2955-66.
Zakarin Safier et al. 2018. Compensating human subjects providing oocytes for stem cell research: 9-year experience and outcomes. J Assist Reprod Genet, 35, 1219-1225.
Zellweger, et al. 2015. Rad51-mediated replication fork reversal is a global response to genotoxic treatments in human cells. J Cell Biol, 208, 563-79.
Zou and Elledge 2003. Sensing DNA damage through ATRIP recognition of RPA-ssDNA complexes. Science, 300, 1542-8.
Zuazua-Villar, et al. 2015. Extensive RPA2 hyperphosphorylation promotes apoptosis in response to DNA replication stress in CHK1 inhibited cells. Nucleic Acids Res, 43, 9776-87.

1. A method of reducing or decreasing replication abnormalities and/or aneuploidies and/or increasing genome stability and/or developmental potential in an embryo comprising introducing into an oocyte at least one of the following:
(a) one or more polynucleotides selected from the group consisting of CHEK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RBI, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof,
(b) one or more polypeptides selected from the group consisting of CHK1, WEE1, ETAA1, ATRX, BLM, BRCA2, CHD4, DNA2 (DNA2L), EXO1, FANCC, FANCG, FBH1 (FBXO18), HLTF (SMARCA3), MCM9, MSH6, POLD3, POLK, RAD51, RAD52, RAD54L, RBI, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof, and
(c) one or more agents that activate and/or increase the expression of one or more genes selected from the group consisting of CHEK1, WEE1, ETAA1, ATRX, BLM, BRCA2, CHD4, DNA2 (DNA2L), EX01, FANCC, FANCG, FBH1 (FBX018), HLTF (SMARCA3), MCM9, MSH6, POLD3, POLK, RAD51, RAD52, RAD54L, RBI, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, and combinations thereof.

2. The method of claim 1, wherein the one or more polynucleotides are mRNA.

3. The method of claim 1, wherein the one or more polynucleotides are introduced into the oocyte using microinjection at the time sperm is introduced into the oocyte during an in vitro fertilization procedure.

4. The method of claim 1, wherein the one or more polypeptides are introduced into the oocyte using microinjection at the time sperm is introduced into the oocyte during an in vitro fertilization procedure.

5. The method of claim 1, wherein the one or more agents are introduced into the oocyte using microinjection at the time sperm is introduced into the oocyte during an in vitro fertilization procedure.

6. A method of reducing or decreasing replication abnormalities and/or aneuploidies and/or increasing genome stability and/or developmental potential in an embryo comprising introducing into an oocyte one or more polynucleotides, polypeptides, and/or agents, wherein the one or more polynucleotides, polypeptides, and/or agents activate the signaling pathway of one or more kinases selected from the group consisting of ATR, WEE1, and CHK1.

7. The method of claim 6, wherein the one or more polynucleotides encode one or more kinases selected from the group consisting of ATR, WEE1, and CHK1.

8. The method of claim 6, wherein the one or more polynucleotides are mRNA.

9. The method of claim 6, wherein the one or more polynucleotides are introduced into the oocyte using microinjection at the time sperm is introduced into the oocyte during an in vitro fertilization procedure.

10. The method of claim 6, wherein one or more polypeptides activate the signaling pathway of one or more kinases selected from the group consisting of ATR, WEE1, and CHK1.

11. The method of claim 10, wherein the one or more polypeptides are selected from the group consisting of ATR, WEE1, and CHK1.

12. The method of claim 10, wherein the one or more polypeptides are introduced into the oocyte using microinjection at the time sperm is introduced into the oocyte during an in vitro fertilization procedure.

13. The method of claim 6, wherein one or more agents activate the signaling pathway of one or more kinases selected from the group consisting of ATR, WEE1, and CHK1.

14. The method of claim 13, wherein the one or more agents are introduced into the oocyte using microinjection at the time sperm is introduced into the oocyte during an in vitro fertilization procedure.

15. A kit comprising one or more polynucleotides and/or polypeptides, and/or agents, wherein the one or more polynucleotides and/or polypeptides and/or agents
(a) activate the signaling pathway of one or more kinases selected from the group consisting of ATR, WEE1, and CHK1; and/or
(b) activate and/or increase expression of genes, selected from the group consisting of CHEK1, WEE1, ETAA1, ATRX, BLM, BRCA2, CHD4, DNA2 (DNA2L), EX01, FANCC, FANCG, FBH1 (FBX018), HLTF (SMARCA3), MCM9, MSH6, POLD3, POLK, RAD51, RAD52, RAD54L, RBI, RECQL, REV3L, RIF1, RNF8, SETD1A, SHPRH, SMARCAL1, TDRD3, TOPBP1, TP53BP1, WRNIP1, XRCC2, WRN, BRCA1, ZRANB3, CDC6, CDT1, POLH, POLI, FANCD2, INO80, FANCB, ASH2L, FAM35A, XRCC3, BRIP1, RNF168, variants, mutants, fragments, derivatives, homologues, or paralogues thereof, and combinations thereof and combinations thereof.

16. The kit of claim 15, further comprising additional reagents selected from the group consisting of reagents for delivery of the polynucleotides, polypeptides and/or agents to an oocyte and for culturing and testing resulting embryos.

17. The kit of claim 15, wherein the one or more polynucleotides, polypeptides and/or agents activate the signaling pathway of one or more kinases selected from the group consisting of ATR, WEE1, and CHK1.

18. The kit of claim 17, further comprising additional reagents selected from the group consisting of reagents for delivery of the polynucleotides, polypeptides and/or agents to an oocyte and for culturing and testing resulting embryos.
